(19) United States
(12) Patent Application Publication
(10) Pub. No.: US 2022/0275406 A1
(43) Pub. Date: Sep. 1, 2022

(54) Membrane Transport Protein and Uses Thereof

(71) Applicant: Oxford University Innovation Limited, Botley, Oxfordshire (GB)

(72) Inventors: Steven KELLY, Botley, Oxfordshire (GB); Michael NIKLAUS, Botley, Oxfordshire (GB); Oliver MATTINSON, Botley, Oxfordshire (GB); Basel ABU-JAMOUS, Botley, Oxfordshire (GB)

(21) Appl. No.: 17/631,846

(22) PCT Filed: Aug. 14, 2020
(86) PCT No.: PCT/IB2020/057658

§ 371 Date: Jan. 31, 2022

(30) Foreign Application Priority Data
Aug. 14, 2019 (AU) 2019902940

(51) Int. Cl. C12P 7/40; C12N 15/82; C12N 15/62; C07K 14/245; C07K 14/415; C12P 7/62

(52) U.S. Cl. CPC C12P 7/40; C12N 15/8243; C12N 15/625; C07K 14/245; C07K 14/415; C12P 7/62; C12N 2800/10

(57) ABSTRACT

Recombinant cells expressing membrane transport proteins are provided, along with methods for their use in various applications. These applications include, without limitation, industrial biotechnology and the reproduction/emulation of biochemical pathways or components thereof (e.g. photosynthetic pathways or components thereof). The recombinant cells may be provided as a component of a transgenic organism (e.g. a transgenic plant).

### TECHNICAL FIELD

The present invention relates to the field of biotechnology, and more specifically to compositions and methods for the transport of molecules across biological membranes (e.g. cell membranes, organelle membranes). Recombinant cells expressing membrane transport proteins are provided, along with methods for their use in various applications. These applications include, without limitation, industrial biotechnology and the reproduction/emulation of biochemical pathways or components thereof (e.g. photosynthetic pathways or components thereof). The recombinant cells may be provided as a component of a transgenic organism (e.g. a transgenic plant).

### BACKGROUND

### Transporters

A number of proteins exist that enable the movement of molecules across biological membranes. These are collectively referred to as transporters, and are subcategorized into four different categories: uniporters, symporters, antiporters, and channels according to their mechanism of action. Uniporters transport a single molecule (charged or uncharged) across a biological membrane. A uniporter may use either facilitated diffusion and/or transport along a diffusion gradient, or may transport against a diffusion gradient using an active transport process. Symporters and antiporters are both types of cotransporter that transport multiple molecules at the same time. Symporters transport these molecules in the same direction in relation to each other, while antiporters transport these molecules in the opposite direction in relation to each other. Channels are proteins that form selective pores in biological membranes that allow the passive, bidirectional transit of certain molecules but not others.

### Monocarboxylates, Dicarboxylates and Tricarboxylates

In living cells, monocarboxylates/monocarboxylic acids, dicarboxylates/dicarboxylic acids and tricarboxylates/tricarboxylic acids are key intermediates in primary metabolism as well as essential building blocks of lipids and amino acids (FIG. 1). Although these metabolites are produced continuously during normal cellular growth, they are also consumed continuously by primary metabolic processes such as respiration and amino acid biosynthesis. Thus, these metabolites normally tend not to accumulate to high levels within cells, and cells do not generally secrete or discard them as waste products.

Monocarboxylates/monocarboxylic acids, dicarboxylates/dicarboxylic acids and tricarboxylates/tricarboxylic acids occupy a central position in industrial biotechnology. Like in living systems, these are used as building blocks for a large range of complex chemicals, non-limiting examples of which include polymers, solvents and pharmaceuticals. Thus, there is a high demand for these simple metabolites. Biological production of these metabolites occurs by fermentation from cheaper sugars. The chassis organisms used for bioproduction of these metabolites either naturally, or have been engineered to, accumulate high concentrations within the cell. Consequently, a large component of the cost of biological production of these metabolites is attributable to the process of extracting the metabolite from the cells and subsequently separating it from other cellular contaminants. Thus, a substantial reduction in the cost of production could be achieved if it was possible to specifically export these metabolites from cells during the process of fermentation. While multiple transporters that import these metabolites into cells have been characterised, there is limited information available regarding transporters capable of exporting these metabolites across biological membranes.

For example, there are two known classes of monocarboxylate transporters: 1) those that symport monocarboxylates/monocarboxylic acids with cations (non-limiting examples include the mitochondrial pyruvate carrier, the bile acid sodium symporters and the monocarboxylate transporter families). 2) those that antiport monocarboxylates/monocarboxylic acids in exchange for dicarboxylates/dicarboxylic acids or tricarboxylates/tricarboxylic acids (non-limiting examples include the bacterial MleN dicarboxylate:monocarboxylate antiporter, and CitP tricarboxylate:monocarboxylate antiporter).

There are three known classes of dicarboxylate/dicarboxylic acid transporters: 1) those that import dicarboxylates/dicarboxylic acids in exchange for phosphate, sulfate, or thiosulfate ions (non-limiting examples include the mitochondrial dicarboxylate carrier and related proteins). 2) those that symport dicarboxylates/dicarboxylic acids with cations (non-limiting examples include the bacterial DctA symporters and related proteins). 3) those that antiport dicarboxylates/dicarboxylic acids in exchange for other tricarboxylates/tricarboxylic acids, dicarboxylates/dicarboxylic acids or monocarboxylates/monocarboxylic acids (non-limiting examples include bacterial Dcu (DcuA, DcuB and DcuC) dicarboxylate antiporters and CitT tricarboxylate:dicarboxylate antiporter, and plant DiT dicarboxylate antiporters). In all cases, there is either no net movement of dicarboxylates/dicarboxylic acids (i.e. dicarboxylates/dicarboxylic acids are antiported for other dicarboxylates/dicarboxylic acids, and thus for every one that goes across the membrane one comes back), or there is net influx of dicarboxylates/dicarboxylic acids. There are no known transporters that facilitate the net movement of dicarboxylates/dicarboxylic acids in the efflux direction.

There are two known classes of tricarboxylate/tricarboxylic acid transporters: 1) those that symport tricarboxylates/tricarboxylic acids with cations (non-limiting examples include bacterial CitM and CitH antiporters). 2) those that antiport tricarboxylates/tricarboxylic acids in exchange for other tricarboxylates/tricarboxylic acids, dicarboxylates/dicarboxylic acids or monocarboxylates/monocarboxylic acids (non-limiting examples include the bacterial CitT, fungal Yhm2, and plant TDT tricarboxylate:dicarboxylate antiporters, and bacterial CitP tricarboxylate:monocarboxylate antiporter).

### C₄ Photosynthesis

Most plant species can be classified into three distinct photosynthetic types; the standard C₃ type and two derived types of photosynthesis known as C₄ and CAM. C₄ plants are in general more efficient in capturing CO₂ and creating biomass than C₃ or CAM plants. For example, although C₄ plants only constitute ˜3% of plant species, they are responsible for 25% of terrestrial CO₂ fixation. In addition, many globally important crop and animal feed plants use C₄ photosynthesis. Thus, understanding how C₄ photosynthesis works is important from both ecological and food security perspectives. However, despite more than 50 years of research into the biochemistry of C₄ photosynthesis, a complete biochemical pathway for C₄ photosynthesis has yet to be described. The missing molecular components of the C₄ cycle in most C₄ species are the monocarboxylate/monocarboxylic acid and dicarboxylate/dicarboxylic acid transporters. Specifically, it is unknown how the dicarboxylate malate enters the bundle sheath chloroplast and how the monocarboxylate pyruvate exits the bundle sheath chloroplast (FIG. 2). The transporters that facilitate these metabolite movements are required to engineer C₄ photosynthesis into C₃ plants.

### SUMMARY OF THE INVENTION

A need exists in the art for the identification of protein/s that can be used to facilitate the export of monocarboxylates/monocarboxylic acids, and/or dicarboxylates/dicarboxylic acids, and/or tricarboxylates/tricarboxylic acids, from cells and/or cell organelles. The identification of such protein/s may be advantageous in numerous application/s including, but not limited to, industrial biotechnology (e.g. production of proteins, peptides, metabolites, molecules, compounds and the like), and/or the enhancement of biochemical pathways in cells (e.g. C₄ photosynthesis, CAM photosynthesis and the like).

The present invention addresses at least one need existing in the art by identifying membrane transporter proteins and demonstrating their ability to export monocarboxylates/monocarboxylic acids, and/or dicarboxylates/dicarboxylic acids, and/or tricarboxylates/tricarboxylic acids, from cells.

The present invention also demonstrates the function of the membrane transporter in the C₄ photosynthetic pathway and demonstrates that the protein can be expressed in the chloroplasts of plants.

The present invention relates at least in part to the following embodiments 1-40 below:

Embodiment 1. A recombinant cell engineered to overexpress a UPF0114 family protein as compared to a corresponding wild-type form of the cell, wherein the UPF0114 family protein is encoded by a recombinant nucleic acid sequence stably or transiently introduced into the recombinant cell, and is capable of transporting carboxylates and/or carboxylic acids across a membrane of the recombinant cell.

Embodiment 2. The recombinant cell of embodiment 1, wherein:
the carboxylates comprise any one of:
   (i) monocarboxylates;
   (ii) dicarboxylates; or
   (iii) tricarboxylates; or
   (iv) monocarboxylates and dicarboxylates; or
   (v) monocarboxylates and tricarboxylates; or
   (vi) dicarboxylates and tricarboxylates; or
   (vii) monocarboxylates, dicarboxylates and tricarboxylates;
the carboxylic acids comprise any one of:
   (i) monocarboxylic acids;
   (ii) dicarboxylic acids; or
   (iii) tricarboxylic acids; or
   (iv) monocarboxylic acids and dicarboxylic acids; or
   (v) monocarboxylic acids and tricarboxylic acids; or
   (vi) dicarboxylic acids and tricarboxylic acids; or
   (vii) monocarboxylic acids, dicarboxylic acids and tricarboxylic acids.

Embodiment 3. The recombinant cell of embodiment 1 or embodiment 2, wherein the corresponding wild-type form of the cell does not express the UPF0114 family protein.

Embodiment 4. The recombinant cell of any one of embodiments 1 to 3, wherein the UPF0114 family protein is exogenous to the recombinant cell.

Embodiment 5. The recombinant cell of any one of embodiments 1 to 4, wherein:
the carboxylates comprise any one or more of: malate, pyruvate, succinate, fumarate, α-ketoglutarate, citrate, glycerate-3-phosphate, phosphoenolpyruvate;
the carboxylic acids comprise any one or more of: malic acid, pyruvic acid, succinic acid, fumaric acid, α-ketoglutaric acid, citric acid, 3-phosphoglyceric acid, phosphoenolpyruvic acid.

Embodiment 6. The recombinant cell of any one of embodiments 1 to 5, wherein the UPF0114 family protein is capable of bidirectional transport of the carboxylates and/or carboxylic acids across the membrane.

Embodiment 7. The recombinant cell of any one of embodiments 1 to 6, wherein the membrane is a cytoplasmic membrane. The cytoplasmic membrane may alternatively be referred to as a cell membrane, cell envelope, cell envelope membrane, or plasma membrane. The cytoplasmic membrane may be a double membrane consisting of an outer membrane and an inner membrane.

Embodiment 8. The recombinant cell of any one of embodiments 1 to 6, wherein the membrane is a cell-internal membrane. The cell-internal membrane may be a chloroplast membrane (e.g. inner and/or outer chloroplast envelope membrane/s, chloroplast internal membranes such as the thylakoid membrane), the peroxisomal membrane, or a mitochondrial membrane (e.g. inner and/or outer mitochondrial membrane/s).

Embodiment 9. The recombinant cell of any one of embodiments 1 to 8, wherein the UPF0114 family protein is capable of transporting carboxylates and/or carboxylic acids across a membrane of the recombinant cell against a concentration gradient existing on one side of the membrane.

Embodiment 10. The recombinant cell of any one of embodiments 1 to 9, wherein the UPF0114 family protein is capable of transporting carboxylates and/or carboxylic acids across a membrane of the recombinant cell with a concentration gradient existing on one side of the membrane.

Embodiment 11. The recombinant cell of any one of embodiments 1 to 10, wherein the recombinant cell is a prokaryotic, eukaryotic, archaeal, plant, algal, bacterial, yeast, fungal, animal, mammalian, or synthetic cell.

Embodiment 12. The recombinant cell of any one of embodiments 1 to 11, wherein the recombinant cell is: a recombinant Corynebacterium species, a recombinant Xanthomonas species, a recombinant Escherichia species, a recombinant Bacillus species, a recombinant Clostridium species, a recombinant Lactobacillus species, a recombinant Lactococcus species, a recombinant Streptococcus species, a recombinant Actinomycetes species, a recombinant Streptomyces species, or a recombinant Actinobacillus species.

Embodiment 13. The recombinant cell of any one of embodiments 1 to 12, wherein the recombinant cell is a recombinant Escherichia coli cell.

Embodiment 14. The recombinant cell of embodiment 11 or embodiment 13, wherein:
the carboxylates comprise any one or more of: succinate, pyruvate, fumarate, malate, citrate, phosphoenolpyruvate, α-ketoglutarate, 3-phosphoglycerate;
the carboxylic acids comprise any one or more of: succinic acid, pyruvic acid, fumaric acid, malic acid, citric acid, phosphoenolpyruvic acid, α-ketoglutaric acid, 3-phosphoglyceric acid.

Embodiment 15. The recombinant cell of any one of embodiments 1 to 11, wherein the recombinant cell is a plant cell or an algal cell.

Embodiment 16. The recombinant cell of embodiment 15, wherein the plant cell is: a vascular sheath cell, a bundle sheath cell, a mestome sheath cell, or a mesophyll cell; of a C₃ photosynthetic plant, a CAM photosynthetic plant, or a C₄ photosynthetic plant.

Embodiment 17. The recombinant cell of embodiment 15 or embodiment 16, wherein:
the carboxylates comprise malate and/or pyruvate;
the carboxylic acids comprise malic acid and/or pyruvic acid.

Embodiment 18. The recombinant cell of embodiment 17, wherein the UPF0114 family protein is capable of uptaking malate and/or malic acid into the recombinant cell and exporting pyruvate and/or pyruvic acid from the recombinant cell.

Embodiment 19. The recombinant cell of embodiment 18, wherein said exporting from the recombinant cell is against a concentration gradient.

Embodiment 20. The recombinant cell of any one of embodiments 15 to 19, wherein the recombinant nucleic acid sequence comprises a sequence encoding a targeting peptide targeting the UPF0114 family protein to a chloroplast membrane, a cytoplasmic membrane, a peroxisomal membrane, or a mitochondrial membrane.

Embodiment 21. The recombinant cell of any one of embodiments 1 to 20, wherein the UPF0114 family protein comprises:
(i) a PFAM protein domain UPF0114 (PF03350) amino acid sequence as defined in any one of SEQ ID NOs: 28-37; or
(ii) a PFAM protein domain UPF0114 (PF03350) amino acid sequence having at least: 70%, 75%, 80%, 85%, 87%, 89%, 90%, 91%, 92%, 93%, 94%, 95%, 96%, 97%, 98%, 99%; sequence identity to any one of SEQ ID NOs: 28-37; or
(iii) a homolog, analog, ortholog or paralog of the PFAM protein domain UPF0114 (PF03350) amino acid sequence of (i) or (ii).

Embodiment 22. The recombinant cell of any one of embodiments 15 to 21, wherein the plant cell is from either of:
(i) a genus Oryza plant (e.g. a rice plant);
(ii) a Oryza sativa or Oryza glaberrima plant. Embodiment 23. The recombinant cell of any one of embodiments 15 to 20, wherein the plant cell is from a: Soy (Glycine max), Cotton (Gossypium hirsutum), Oilseed rape/Cannola (B. napus subsp. Napus), Potato (Solanum tuberosum), tomato (Solanum lycopersicum), Cassava (Manihot esculenta), Wheat (Triticum aestivum), Barley (Hordeum vulgare), pigeon pea (Cajanus cajan), cowpea (Vigna unguiculata), pea (Pisum sativum), cannabis (Cannabis sativa), sugar beet (Beta vulgaris), oat (Avena sativa), rye (Secale cereal), peanut (Arachis hypogaea), Sunflower (Helianthus annuus), flax (Linum spp.), beans (Phaseolus vulgaris), lima bean (Phaseolus lunatus), mung bean (Phaseolus mung), Adzuki bean (Phaseolus angularis), Chickpea (Cicer arietinum), tobacco (Nicotiana tabacum), buckwheat (Fagopyrum esculentum), oil palm (Elaeis guineensis), or rubber (Hevea brasiliensis); plant.

Embodiment 24. The recombinant cell of any one of embodiments 1 to 23, wherein the UPF0114 family protein is any one of: a C₄ photosynthetic plant UPF0114 protein, a C₃ photosynthetic plant UPF0114 protein, an algal UPF0114 protein, a bacterial UPF0114 protein, or an archaeal UPF0114 protein.

Embodiment 25. The recombinant cell of any one of embodiments 1 to 24, wherein the UPF0114 family protein is any one of:
(i) an Arabidopsis thaliana UPF0114 protein;
(ii) a Setaria italica UPF0114 protein;
(iii) a Setaria viridis UPF0114 protein;
(iv) an Escherichia coli UPF0114 protein;
(v) a Zea mays UPF0114 protein;
(vi) a UPF0114 protein comprising or consisting of an amino acid sequence having at least: 70%, 75%, 80%, 85%, 87%, 89%, 90%, 91%, 92%, 93%, 94%, 95%, 96%, 97%, 98%, 99%; sequence identity to the UPF0114 protein of (i), (ii), (iii), (iv) or (v); (vii) a homolog, analog, ortholog or paralog of the UPF0114 protein of (i), (ii), (iii), (iv) or (v).

Embodiment 26. The recombinant cell of any one of embodiments 1 to 24, wherein the UPF0114 family protein:
(i) comprises or consists of an amino acid sequence as defined in SEQ ID NO: 1, SEQ ID NO: 2, SEQ ID NO: 3, SEQ ID NO: 4, SEQ ID NO: 5, SEQ ID NO: 6; SEQ ID NO: 9, SEQ ID NO: 10, SEQ ID NO: 11, SEQ ID NO: 15, SEQ ID NO: 18, SEQ ID NO: 19, SEQ ID NO: 20, SEQ ID NO: 21, SEQ ID NO: 212, SEQ ID NO: 23, SEQ ID NO: 24, SEQ ID NO: 25, SEQ ID NO: 26, or SEQ ID NO: 27; or
(ii) comprises or consists of an amino acid sequence having at least: 70%, 75%, 80%, 85%, 87%, 89%, 90%, 91%, 92%, 93%, 94%, 95%, 96%, 97%, 98%, 99%; sequence identity to SEQ ID NO: 1, SEQ ID NO: 2, SEQ ID NO: 3, SEQ ID NO: 4, SEQ ID NO: 5, SEQ ID NO: 6 SEQ ID NO: 9, SEQ ID NO: 10, SEQ ID NO: 11, SEQ ID NO: 15, SEQ ID NO: 18, SEQ ID NO: 19, SEQ ID NO: 20, SEQ ID NO: 21, SEQ ID NO: 212, SEQ ID NO: 23, SEQ ID NO: 24, SEQ ID NO: 25, SEQ ID NO: 26, or SEQ ID NO: 27; or
(iii) is a homolog, analog, ortholog or paralog of the UPF0114 family protein comprising or consisting of an amino acid sequence of (i) or (ii); or
(iv) is encoded by a nucleotide sequence comprising or consisting of SEQ ID NO: 7, SEQ ID NO: 8, SEQ ID NO: 12, SEQ ID NO: 13, SEQ ID NO: 14, or SEQ ID NO: 16; or
(v) is encoded by a nucleotide sequence comprising or consisting a nucleotide sequence having at least: 70%, 75%, 80%, 85%, 87%, 89%, 90%, 91%, 92%, 93%, 94%, 95%, 96%, 97%, 98%, 99%; sequence identity to SEQ ID NO: 7 SEQ ID NO: 8, SEQ ID NO: 12, SEQ ID NO: 13, SEQ ID NO: 14, or SEQ ID NO: 16; or
(vi) is a homolog, analog, ortholog or paralog of the UPF0114 family protein encoded by the nucleotide sequence of (iv) or (v).

Embodiment 27. The recombinant cell of any one of embodiments 1 to 26, wherein the recombinant nucleic acid sequence:
(i) is operably linked to a regulatory sequence; and/or
(ii) is a component of an expression vector; and/or
(iii) is codon optimised for expression in the recombinant cell type; and/or
(iv) has intronic sequences removed; and/or
(v) comprises a signal peptide sequence for directing the UPF0114 family protein to an internal membrane or cytoplasmic membrane of the recombinant cell.

Embodiment 28. The recombinant cell of any one of embodiments 1 to 27, wherein the carboxylates and/or carboxylic acids are phosphorylated.

Embodiment 29. The recombinant cell of any one of embodiments 1 to 28, wherein recombinant cell is further engineered to produce or overexpress an enzyme and/or regulatory protein of a biochemical pathway, for production of the carboxylates and/or carboxylic acids.

Embodiment 30. The recombinant cell of embodiment 29, wherein the recombinant cell comprises an expression vector comprising a further nucleic acid sequence encoding the enzyme and/or the regulatory protein.

Embodiment 31. A transgenic plant or a seed thereof comprising the recombinant cell of any one of embodiments 15 to 30.

Embodiment 32. The transgenic plant of embodiment 31 comprising a gene selected from any one or more of: carbonic anhydrase (CA), phosphoenolpyruvate carboxylase (PEPC), malate dehydrogenase (MDH), oxaloacetate/malate transporter (OMT), NADP malic enzyme (NADP-ME), bile acid sodium symporter 2 (BASS2), pyruvate, phosphate dikinase (PPDK), phosphoenolpyruvate phosphate translocator (PPT).

Embodiment 33. Use of the recombinant cell of any one of embodiments 1 to 30 in a process for producing carboxylic acids and/or carboxylates.

Embodiment 34. A process for production of carboxylic acids and/or carboxylates comprising:
(i) producing the carboxylates in the recombinant cell according to any one of embodiments 1 to 30, and
(ii) exporting the carboxylates from the recombinant cell using a UPF0114 family protein embedded within the membrane of the recombinant cell.

Embodiment 35. The process of embodiment 34, further comprising isolating the carboxylic acids and/or carboxylates when exported from the UPF0114 family protein.

Embodiment 36. The process of embodiment 34 or embodiment 35, wherein the UPF0114 family protein exports the carboxylic acids and/or carboxylates against a concentration gradient.

Embodiment 37. The process of any one of embodiments 34 to 36, wherein the carboxylic acids and/or carboxylates are produced in the recombinant cell using an expression vector comprising a nucleic acid sequence encoding an enzyme and/or regulatory protein of a biochemical pathway for production of the carboxylic acids and/or carboxylates.

Embodiment 38. The process of any one of embodiments 34 to 37, wherein the carboxylic acids and/or carboxylates are produced in the recombinant cell by uptake of one or more carboxylic acids and/or carboxylate precursors into the recombinant cell, and conversion of the precursors into the carboxylic acids and/or carboxylates within the recombinant cell.

Embodiment 39. The process of embodiment 38, wherein the uptake of the one or more carboxylic acids and/or carboxylates precursors occurs via the UPF0114 family protein.

Embodiment 40. The process of any one of embodiments 34 to 39, wherein:
the carboxylates comprise any one or more of: malate, pyruvate, succinate, fumarate, α-ketoglutarate, citrate, glycerate-3-phosphate, phosphoenolpyruvate;
the carboxylic acids comprise any one or more of: malic acid, pyruvic acid, succinic acid, fumaric acid, α-ketoglutaric acid, citric acid, 3-phosphoglyceric acid, phosphoenolpyruvic acid.

### DEFINITIONS

As used in this application, the singular form “a”, “an” and “the” include plural references unless the context clearly dictates otherwise. For example, the term “cell” also includes multiple cells unless otherwise stated.

As used herein, the term “comprising” means “including”. Variations of the word “comprising”, such as “comprise” and “comprises” have correspondingly varied meanings. Thus, for example, a polynucleotide “comprising” nucleotide sequence ‘A’ may consist exclusively of nucleotide sequence ‘A’, or may include one or more additional nucleotide sequence/s, for example, nucleotide sequence ‘B’ and/or nucleotide sequence ‘C’.

As used herein, a “carboxylate” is a salt or ester of a carboxylic acid. A “carboxylic acid” includes any organic compound that has one, two or three carboxylic acid functional groups.

As used herein, a “monocarboxylate” is a salt or ester of a monocarboxylic acid. A “monocarboxylic acid” is any organic compound that has one carboxylic acid functional group.

As used herein, a “dicarboxylate” is a salt or ester of a dicarboxylic acid. A “dicarboxylic acid” is any organic compound that has two carboxylic acid functional groups.

As used herein, a “tricarboxylate” is a salt or ester of a tricarboxylic acid. A “tricarboxylic acid” is any organic compound that has three carboxylic acid functional groups.

As used herein, a “recombinant cell” will be understood to mean a cell into which a recombinant nucleic acid (e.g. recombinant DNA, recombinant RNA) has been introduced. A “recombinant nucleic acid” is a nucleic acid sequence comprising a combination of nucleic acid molecules that would not otherwise exist in nature. Recombinant nucleic acids as referred to herein may be synthesised recombinant nucleic acids.

As used herein, a “UPF0114 protein”, will be understood to refer to a transmembrane protein comprising at least one sequence corresponding to PFAM protein domain UPF0114 (PF03350), a characteristic domain of the UPF0114 family that comprises transmembrane helices (e.g. three to four). Non-limiting examples of PFAM protein domain UPF0114 (PF03350) sequences are provided in SEQ ID NOs: 28-37, and further non-limiting examples include any one or more of homologs, analogs, orthologs and/or paralogs of the sequences provided in SEQ ID NOs: 28-37. A protein can be identified as a “UPF0114 protein” when its amino acid sequence produces a statistically significant hit (i.e. an E-value≤0.001) when aligned to the profile hidden Markov model* for the domain PFAM domain PF03350 (*see, for example, Eddy, S R. (1998) Profile hidden Markov models. Bioinformatics 14:755-763; and Finn, R D. (2015) The Pfam protein families database: towards a more sustainable future. Nucleic Acids Research 44:D279-85). A “UPF0114 protein” may comprise additional domain(s) including, for example, one or more AAA+ATPase domains, one or more ATP-binding domains, one or more nucleotide triphosphate hydrolase domains, one or more SHOCT domains, one or more Fe-S hydro-lyase domains, one or more NB-ARC domains, one or more cytochrome C oxidase domains, one or more reverse transcriptase domains, one or more structural maintenance of chromosomes domains, and/or one or more major facilitator superfamily domains. “UPF0114 protein(s)” may also be referred to herein as “UPF0114 family protein(s)”, proteins of the “UPF0114 protein family”, or “member(s) of the UPF0114 protein family”, and may exist, for example, in any of viruses, bacteria, archaea, algae, and plants.

As used herein, a “PFAM” protein will be understood to be a constituent of the Pfam database (e.g. Pfam 33.1) — see https://pfam.xfam.org/; El-Gebali et al. (2019) “The Pfam protein families database in 2019”, Nucleic Acids Research doi: 10.1093/nar/gky995. The data presented for a given PFAM protein entry is based on the UniProt Reference Proteomes, but information on individual UniProtKB sequences can still be found by entering the protein accession. Pfam full alignments are available from searching a variety of databases, either to provide different accessions (e.g. all UniProt and NCBI GI) or different levels of redundancy.

As used herein, a “cytoplasmic membrane” will be understood to mean a biological membrane that separates the interior of a cell from its external environment. Other terms used herein and/or in the art which will be understood to be equivalent to “cytoplasmic membrane” include “cell membrane”, “cell envelope”, “cell envelope membrane”, and “plasma membrane”. In the cases where cells have double membranes, the term “cytoplasmic membrane” will be understood herein to include the outer and/or inner membrane/s of the cell.

As used herein, the terms “overexpress”, “overexpressed” and “overexpression” in the context of expressing a given biological entity (e.g. nucleic acid, protein, peptide and the like) in a recombinant cell refers to: (i) expression of the entity in the recombinant cell at a level greater than a level of expression of the same entity in a corresponding wild-type cell; or (ii) expression of the entity in the recombinant cell at a detectable level when a corresponding wild-type cell expresses the same entity at detectable levels, or does not express the entity at all.

As used herein, the term “corresponding wild-type” in the context of modified cells, organisms, nucleic acid sequences, proteins, peptides and the like refers to the natural form of the entity. For example, in the case of a recombinant cell engineered to contain a vector comprising an exogenous nucleic acid sequence, the “corresponding wild-type” cell would be the cell as it existed in natural form prior to having been engineered to include the vector. By way of further non-limiting example, the “corresponding wild-type” of a codon-optimised nucleic acid or amino acid sequence would be the sequence as it existed in natural form prior to the codon optimisation.

As used herein, a “C₃ photosynthetic plant”, will be understood to encompass any plant in which all or the majority of photosynthesis is limited to C₃ photosynthesis. “C₃ photosynthesis” means a photosynthetic pathway which uses only the Calvin-Benson cycle for fixing carbon dioxide from air, providing a three-carbon compound. Cell types referred to herein as “C₃” will be understood to be from a “C₃ photosynthetic plant”.

As used herein, a “C₄ photosynthetic plant” will be understood to encompass any plant in which all or the majority of photosynthesis is limited to C₄ photosynthesis. “C₄ photosynthesis” means a photosynthetic pathway in which an intermediate four-carbon compound is used to transfer CO₂ to the site of CO₂ fixation through the Calvin-Benson cycle. C₄ photosynthesis commences with light-dependent reactions in mesophyll cells and the preliminary fixation of carbon dioxide to malate. Carbon dioxide is released from malate, where it is fixed again by RuBisCO and the Calvin-Benson cycle. Cell types referred to herein as “C₄” will be understood to be from a “C₄ photosynthetic plant”. C₄ photosynthesis can occur in a single cell or can be distributed across multiple cells in a plant leaf.

As used herein, a “CAM photosynthetic plant” will be understood to encompass any plant in which all or the majority of the photosynthetically active tissues of the plant conduct CAM photosynthesis. “CAM photosynthesis” is also known as “crassulacean acid metabolism” and means a photosynthetic pathway that comprises a temporally distributed carbon fixation pathway. In plants that conduct CAM photosynthesis the stomata are open at night to allow CO₂ to diffuse in to the leaf and be fixed into C₄ acids by the enzyme phosphoenolpyruvate carboxylase. These C₄ acids accumulate during the night and then during the day the plants close their stomata and decarboxlate the C₄ acids to release CO₂ around RuBisCO. Thus, PEP carboxylation and RuBisCO carboxylation are temporally separated in CAM plants. “CAM photosynthetic plants” as referred to herein include “inducible CAM plants” or “facultative CAM plants”, which will be understood to be plants that can switch between normal C₃ photosynthesis and CAM photosynthesis depending on environmental conditions. The “inducible CAM plants” may also switch between CAM and C₄ photosynthesis. “CAM photosynthetic plants” as referred to herein may also conduct a version of CAM photosynthesis known as “CAM-cycling”, in which stomata do not open at night, but instead the plants recycle CO₂ produced by respiration and store some CO₂ that is captured during the day.

As used herein, the term “carboxylate/carboxylic acid” will be understood to mean carboxylate and/or carboxylic acid.

As used herein, the term “monocarboxylate/monocarboxylic acid” will be understood to mean monocarboxylate and/or monocarboxylic acid.

As used herein, the term “dicarboxylate/dicarboxylic acid” will be understood to mean dicarboxylate and/or dicarboxylic acid.

As used herein, the term “tricarboxylate/tricarboxylic acid” will be understood to mean tricarboxylate and/or tricarboxylic acid.

As used herein, the phrase “against a concentration gradient” in the context of transporting a molecule across a biological membrane is intended to mean that the molecule is transported from a first location adjacent to one side of the membrane having a first concentration (number of molecules/unit of solute) to a second location adjacent to an opposing side of the membrane which has a second concentration (number of molecules/unit of solute) of the molecule, wherein the second concentration is higher than the first concentration.

As used herein, a percentage of “sequence identity” will be understood to arise from a comparison of two sequences in which they are aligned to give a maximum correlation between the sequences. This may include inserting “gaps” in either one or both sequences to enhance the degree of alignment. The percentage of sequence identity may then be determined over the length of each of the sequences being compared. For example, a nucleotide sequence (“subject sequence”) having at least 95% “sequence identity” with another nucleotide sequence (“query sequence”) is intended to mean that the subject sequence is identical to the query sequence except that the subject sequence may include up to five nucleotide alterations per 100 nucleotides of the query sequence. In other words, to obtain a nucleotide sequence of at least 95% sequence identity to a query sequence, up to 5% (i.e. 5 in 100) of the nucleotides in the subject sequence may be inserted or substituted with another nucleotide or deleted.

As used herein, a regulatory sequence “operably linked” to another sequence means that a functional relationship exists between the two sequences such that the regulatory sequence has the capacity to exert an influence on the expression and/or localisation and/or activity of the sequence to which it is linked. For example, a promoter operably linked to a coding sequence will be capable of modulating the transcription of the coding sequence. A targeting peptide operably linked to a polypeptide will be capable of directing the polypeptide to a specific location (e.g. an organelle or cytoplasmic membrane).

### BRIEF DESCRIPTION OF THE FIGURES

Preferred embodiments of the present invention will now be described by way of example only, with reference to the accompanying figures wherein:

FIG. 1 depicts the tricarboxylic acid cycle (citrate cycle) in E. coli.

FIG. 2 depicts the current understanding of the C₄ photosynthetic cycle. Transporters located in the chloroplast envelope are indicated by two blue circles. Gene names are indicated by bold blue text. The missing transporters of the C₄ cycle are indicated by red circles and red font question marks (???). CA: carbonic anhydrase. PEPC: phosphoenolpyruvate carboxylase. MDH: malate dehydrogenase. OMT: oxaloacetate/malate transporter. CBC: Calvin-Benson Cycle. NADP-ME: NADP malic enzyme. BASS2: bile acid sodium symporter. PPDK: pyruvate, phosphate dikinase. PPT: phosphoenolpyruvate phosphate translocator. OAA: oxaloacetate. MAL: malate. PYR: pyruvate. PEP phosphoenolpyruvate.

FIG. 3 depicts non-limiting set of dicarboxylate/dicarboxylic acid metabolites that are transported by transporters of the present invention. The dicarboxylate/dicarboxylic acid is indicated on the y-axis label. Non-Ind denotes the abundance of the metabolite in the cell culture supernatant of the E. coli cell line with no transporter expression. Si Ind denotes the abundance of the metabolite in the cell culture supernatant when the protein encoded by the Sevir.4G287300 gene from Setaria viridis is expressed. At Ind denotes the abundance of the metabolite in the cell culture supernatant when the protein encoded by the AT4G19390 gene from Arabidopsis thaliana is expressed. (μM) means micromolar. Cells were grown in M9 minimal medium with glucose as a sole carbon source.

FIG. 4 depicts non-limiting examples of monocarboxylate/monocarboxylic acid metabolites that are transported by transporters of the present invention. The monocarboxylate/monocarboxylic acid is indicated on the y-axis label. Non-Ind denotes the abundance of the metabolite in the cell culture supernatant of the E. coli cell line with no transporter expression. Si Ind denotes the abundance of the metabolite in the cell culture supernatant when the protein encoded by the Sevir.4G287300 gene in Setaria viridis is expressed. At Ind denotes the abundance of the metabolite in the cell culture supernatant when the protein encoded by the AT4G19390 gene from Arabidopsis thaliana is expressed. (μM) means micromolar. Cells were grown in M9 minimal medium with glucose as a sole carbon source.

FIG. 5 depicts non-limiting examples of tricarboxylate/tricarboxylic acid metabolites that are transported by transporters of the present invention. The tricarboxylate/tricarboxylic acid is indicated on the y-axis label. Non-Ind denotes the abundance of the metabolite in the cell culture supernatant of the E. coli cell line with no transporter expression. Si Ind denotes the abundance of the metabolite in the cell culture supernatant when the protein encoded by the Sevir.4G287300 gene in Setaria viridis is expressed. At Ind denotes the abundance of the metabolite in the cell culture supernatant when the protein encoded by the AT4G19390 gene from Arabidopsis thaliana is expressed. (μM) means micromolar. Cells were grown in M9 minimal medium with glucose as a sole carbon source.

FIG. 6 depicts non-limiting examples of phosphorylated carboxylate metabolites that are transported by transporters of the present invention. The metabolite is indicated on the y-axis label. Non-Ind denotes the abundance of the metabolite in the cell culture supernatant of the E. coli cell line with no transporter expression. Si Ind denotes the abundance of the metabolite in the cell culture supernatant when the protein encoded by the Sevir.4G287300 gene from Setaria viridis is expressed. At Ind denotes the abundance of the metabolite in the cell culture supernatant when the protein encoded by the AT4G19390 gene from Arabidopsis thaliana is expressed. (μM) means micromolar. 3-PGA means 3-Phosphoglyceric acid (3PG) which is the conjugate acid of glycerate 3-phosphate. Cells were grown in M9 minimal medium with glucose as a sole carbon source.

FIG. 7 depicts a non-limiting example of how a transporter protein of the present invention can export metabolites to a higher concentration than the intracellular concentration of the metabolite. Here expression of the Setaria viridis version of the transporter was induced at time 0 with three different starting concentrations of pyruvate. The intracellular concentration of pyruvate in E. coli was 390 μM; this concentration is indicated by a dashed horizontal red line. Cells were grown in M9 minimal medium with glucose as a sole carbon source.

FIG. 8 depicts the pyruvate export activity of the transporter encoded by the E. coli yqhA gene of the present invention. The y-axis depicts the concentration of pyruvate measured in the cell culture supernatant of the non-induced cells (Non-ind) and the cells expressing the transporter (yqhA ind). Cells were grown in M9 minimal medium with glucose as a sole carbon source.

FIG. 9 depicts a non-limiting example of the bidirectional transport activity of a transporter protein of the present invention. Here an E. coli strain has been engineered to delete the endogenous dicarboxylate/dicarboxylic acid import protein DctA (AdctA). Thus, this cell line cannot import any dicarboxylates/dicarboxylic acids and thus cannot grow on dicarboxylates/dicarboxylic acids as a sole carbon source. Here expression of the protein encoded by the Sevir.4G287300 gene from Setaria viridis was induced at time 0 in the presence or absence of malate as a sole carbon source. Export of pyruvate to the cell culture medium demonstrates that the transporter can both uptake malate and export pyruvate. This is exactly the transport reaction required by the bundle sheath cell chloroplast of NADP-ME C₄ plants to conduct C₄ photosynthesis.

FIG. 10 depicts the relative abundance of the transcripts corresponding to the Sevir.4G287300 gene in Setaria viridis in wild-type plants and in stably transformed plants that have been engineered to contain an RNAi construct that targets the RNAi mediated downregulation of transcripts corresponding to the same gene. The y-axis is in arbitrary units. Relative transcript abundance for wild-type plants is on the left and relative transcript abundance for Sevir.4G287300 RNAi plants is on the right.

FIG. 11 depicts the effect on photosynthesis of RNAi mediated downregulation of Sevir.4G287300 in Setaria viridis. This shows that photosynthesis is severely reduced in the mutant lines (grey dots, labelled “Transporter RNAi lines” in the figure) compared to azygous lines from the same transformation events. The azygous (black dots labelled “Segregating wild-type lines” in the figure) lines are progeny of transgenic parent lines that have lost the transgene through segregation. Azygous plants are considered ideal controls because they have been through the entire process of generating transgenic plants, exactly like their transgenic “sibling” plants. The graph shows photosynthetic carbon assimilation rate (A) plotted as a function of sub-stomatal CO₂ concentration (Ci).

FIG. 12 depicts a complete C₄ cycle. This C₄ cycle utilises a transporter protein of the present invention (labelled in red as CTP1 for Carboxylate transport protein 1). This protein can be any member of the UPF0114 protein family. CA: carbonic anhydrase. PEPC: phosphoenolpyruvate carboxylase. MDH: malate dehydrogenase. OMT: oxaloacetate/malate transporter. CBC: Calvin Benson Cycle. NADP-ME: NADP malic enzyme. BASS2: bile acid sodium symporter. PPDK: pyruvate, phosphate dikinase. PPT: phosphoenolpyruvate phosphate translocator. OAA: oxaloacetate. MAL: malate. PYR: pyruvate. PEP: phosphoenolpyruvate.

FIG. 13 depicts the localisation of the Arabidopsis thaliana AT4G19390::GFP C-terminal translational fusion in Arabidopsis thaliana leaf protoplasts. The localisation of GFP is provided as a control.

FIG. 14 depicts the localisation of the Setaria italica Si007164m::GFP C-terminal translational fusion in Setaria viridis leaf protoplasts. The localisation of GFP is provided as a control.

FIG. 15 depicts the pANIC 12A RNAi vector used to knock-down the expression of the Setaria viridis Sevir.4G287300 gene.

FIG. 16 depicts the mRNA abundance of the Setaria viridis Sevir.4G287300 gene in bundle sheath cells and mesophyll cells of mature leaves in Setaria viridis plants. TPM is transcripts per million transcripts.

FIG. 17 depicts the growth of ΔdctA E. coli lines on M9 minimal medium supplemented with different carbon sources. ΔdctA E. coli cells grow on M9 glucose, but as ΔdctA E. coli cells cannot import the dicarboxylate malate, they cannot grow on malate as a sole carbon source. Wild-type cells can import the dicarboxylate malate, and thus they grow on M9 supplemented with malate as a sole carbon source. T0 is the timepoint at the start of an induction. T1 is 36 hours after T0.

FIG. 18 depicts the E. coli inducible expression vector used for expressing the transgenes used in this study. The example shown here includes the Escherichia coli codon optimised version of the Setaria italica Si007164m (Seita.4G275500) gene with no chloroplast target peptide. The amino acid sequence of the Setaria italica gene is 100% identical to that of the Setaria viridis gene Sevir.4G287300.

FIG. 19 depicts the pyruvate export activity of the transporter proteins encoded by the Zea mays GRMZM2G327686, GRMZ2G133400 and GRMZM2G179292 genes of the present invention. The y-axis depicts the concentration of pyruvate measured in the cell culture supernatant of the non-induced cells (−) and the cells expressing the transporter (+). Cells were grown in M9 minimal medium with glucose as a sole carbon source.

FIG. 20 depicts the localisation of the Setaria italica Si007164m::GFP C-terminal translational fusion in Oryza sativa leaf protoplasts. The localisation of GFP is provided as a control.

FIG. 21 depicts pyruvate export activity of the transporter protein encoded by the Setaria italica Si007164m gene (SEQ ID NO: 8) when expressed in E. coli in the presence of different four-carbon dicarboxylates in the cell culture medium.

FIG. 22 A) depicts the mRNA abundance of the Talinum triangulare gene Tt48731 which is the ortholog of AT4G19390, Sevir.4G287300 and Seita.4G275500. B) depicts the mRNA abundance of the Talinum triangulare gene Tt38957, that encodes chloroplast localized NADP-ME-2. In both cases, mRNA abundance is measured during a CAM induction cycle, wherein the plant is deprived of water for 12 days to cause the plant to switch from C₃ photosynthesis to CAM photosynthesis. The plants switch by day 9. Following day 12, the plants are re-watered and the plants revert back to C₃ photosynthesis within 2 days.

FIG. 23 depicts the localisation of the Arabidopsis thaliana AT4G19390::GFP C-terminal translational fusion expressed in leaf cells of Nicotiana benthamiana. Two example images are shown to depict the localisation to the chloroplast envelope. The localisation of GFP is provided as a control. Scale bar =5μm.

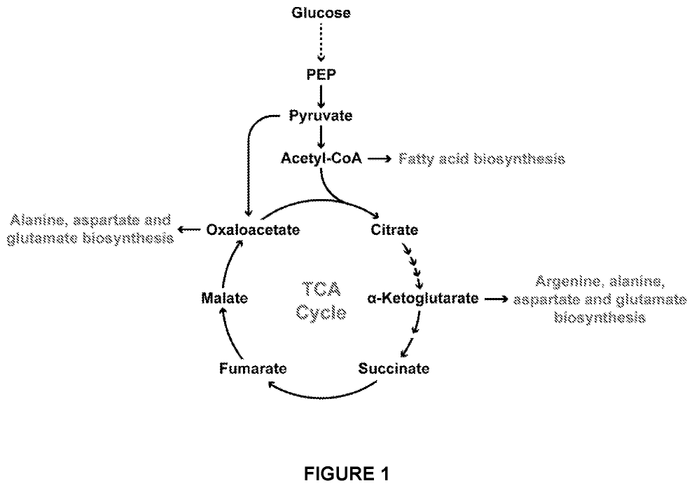

### DETAILED DESCRIPTION

The following detailed description conveys exemplary embodiments of the present invention in sufficient detail to enable those of ordinary skill in the art to practice the present invention. Features or limitations of the various embodiments described do not necessarily limit other embodiments of the present invention, or the present invention as a whole. Hence, the following detailed description does not limit the scope of the present invention, which is defined only by the claims.

It will be appreciated by persons of ordinary skill in the art that numerous variations and/or modifications can be made to the present invention as disclosed in the specific embodiments without departing from the spirit or scope of the present invention as broadly described. The present embodiments are, therefore, to be considered in all respects as illustrative and not restrictive.

Known transporters of monocarboxylates, dicarboxylates and tricarboxylates are suboptimal for many applications in industrial biotechnology due to their inability to export these molecules from the cells in which they are produced or overexpressed. This adds to the complexity, time and/or cost of processes aimed at the mass production of these metabolites. Additionally, although the C₄ photosynthetic pathway is well-characterised, the missing/unknown molecular components of the C₄ cycle in most C₄ species are the monocarboxylate/monocarboxylic acid and dicarboxylate/dicarboxylic acid transporters. Specifically, in C₄ plants it is unknown how the dicarboxylate malate enters the bundle sheath chloroplast and how the monocarboxylate pyruvate exits the bundle sheath chloroplast.

The present inventors have identified that UPF0114 family proteins provide a means of transporting monocarboxylates/monocarboxylic acids, and/or dicarboxylates/dicarboxylic acids, and/or tricarboxylates/tricarboxylic acids, across cell membranes (internal and/or external), and in particular a means of exporting these molecules from cells into the external environment. In doing so, they have provided a solution to current difficulties experienced in isolating these molecules from cells in the industrial biotechnology setting.

Additionally, as noted above the identity of the transporters facilitating movement of the dicarboxylate malate into the bundle sheath chloroplast and the exit of the monocarboxylate pyruvate from the bundle sheath chloroplast is needed to engineer C₄ photosynthesis into C₃ plants. The present inventors have demonstrated that UPF0114 family proteins from C₄ photosynthetic plants facilitate both uptake of malate and export of pyruvate, as required for the bundle sheath cell chloroplast to conduct C₄ photosynthesis. They have also shown that reduction of the amount of transcript encoding the UPF0114 protein in the C₄ plant Setaria viridis, severely disrupts C₄ photosynthesis and thus that the UPF0114 family protein is required for C₄ photosynthesis. They have additionally shown that UPF0114 family proteins can be over-expressed in both C₃ and C₄ plant cells including rice (Oryza sativa).

### UPF0114 Protein Family

The present invention provides recombinant cells expressing UPF0114 family proteins, and methods and processes for using them.

Prior to the present invention, the UPF0114 protein family (also known as the yqhA gene family) had not been functionally characterized and its biological role was unknown. Genes encoding members of the UPF0114 protein family can be found in the genomes of viruses, bacteria, archaea, algae, plants and some other eukaryotic organisms, and are defined by the presence of the PFAM protein domain of the same name; UPF0114 (PF03350). This PFAM domain typically comprises three or four transmembrane helices. Members of the UPF0114 protein family may comprise additional domains in addition to the UPF0114 domain. Non-limiting examples include any one or more: AAA+ATPase domains, ATP-binding domains, nucleotide triphosphate hydrolase domains, SHOCT domains, Fe-S hydro-lyase domains, NB-ARC domains, cytochrome C oxidase domains, reverse transcriptase domains, structural maintenance of chromosomes domains, major facilitator superfamily domains. Members of the UPF0114 protein family may also comprise a chloroplast and/or a mitochondrial targeting peptide (e.g. algae and plant UPF0114 family proteins). Non-limiting/representative UPF0114 protein family sequences from various organisms including viruses, archaea, bacteria, green algae and plants (SEQ ID NOs: 18-27) and their individual PFAM domain PF03350 sequences (SEQ ID NOs: 28-37) are provided below.

A non-limiting example of a viral protein in the UPF0114 family is the AXQ68784.1 protein in the Caulobacter phage CcrPW. The UPF0114 PFAM domain PF03350 is shown underneath.

(SEQ ID NO: 18)
MIFETRWLLVPIYLAMIIAIAAYVILFTKQAIDMG
LGVWHWDAEHLLLASLALVDMSMVANLIVMILAGG
FSTFVAEFDQSLFPNRPRWMNGLDSTTLKIQMGKS
LIGVTSVHLLQTFMRLHDILKEENGLVLVIAEIAI
HMVFIVTTVSYCYISKLTHGHKVAPAALPTPATAE
GH

Caulobacter phage CcrPW AXQ68784.1 protein PFAM domain PF03350 sequence:

(SEQ ID NO: 28)
IFETRWLLVPIYLAMIIAIAAYVILFTKQAIDMGL
GVWHWDAEHLLLASLALVDMSMVANLIVMILAGGF
STFVAEFDQSLFPNRPRWMNGLDSTTLKIQMGKSL
IGVTSVHLLQTFMRLHDILKEENGLVLVIAEIA

A non-limiting example of an archaeal protein in the UPF0114 family is the WP_095643983.1 protein in Methanosarcina spelaei. The UPF0114 domain is shown underneath.

(SEQ ID NO: 19)
MKVVRFIAGMRFFVLIPVIGLAIAACVLFIKGGID
IIHFMGELIIGMSEEGPEKSIIVEIVETVHLFLVG
TVLFLTSFGLYQLFIQPLPLPEWVKVNNIEELELN
LVGLTVVVLGVNFLSIIFEPQETDLAIYGIGYALP
IAALAYFMKVRSHIRKGSNDEEEMRNIGEVTSVNS
ESNWLINKKGD

Methanosarcina spelaei WP_095643983.1 protein PFAM domain PF03350 sequence:

(SEQ ID NO: 29)
VVRFIAGMRFFVLIPVIGLAIAACVLFIKGGIDII
HFMGELIIGMSEEGPEKSIIVEIVETVHLFLVGTV
LFLTSFGLYQLFIQPLPLPEWVKVNNIEELELNLV
GLTVVVLGVNFLSIIFEPQETDLAIYGIGYALPIA
ALAYF

Another non-limiting example of an archaeal protein in the UPF0114 family is the WP_012192968.1 protein in Methanococcus maripaludis. The UPF0114 PFAM domain PF03350 is shown underneath.

(SEQ ID NO: 20)
MGKSDKLKKKYGIKNISEQGFFEHFFELILWNSRF
IVVLAVIFGTLGSIMLFLAGSAEIFHTILSYISDP
MSSEQHNQILIGVIGAVDLYLIGVVLLIFSFGIYE
LFISKIDIARVDGDVSNILEIYTLDELKSKIIKVI
IMVLVVSFFQRVLSMHFETSLDMIYMAISIFAISL
GVYFMHRQKM

Methanococcus maripaludis WP_012192968.1 protein PFAM domain PF03350 sequence:

(SEQ ID NO: 30)
FEHFFELILWNSRFIVVLAVIFGTLGSIMLFLAGSAEIFHTILSYISDPM
SSEQHNQILIGVIGAVDLYLIGVVLLIFSFGIYELFISKIDIARVDGDVS
NILEIYTLDELKSKIIKVIIMVLVVSFFQRVLSMHFETSLDMIYMAISIF
AISLGVYFM

A non-limiting example of a bacterial protein in the UPF0114 family is the yqhA protein in Escherichia coli. The UPF0114 PFAM domain PF03350 is shown underneath.

(SEQ ID NO: 21)
MERFLENAMYASRWLLAPVYFGLSLALVALALKFFQEIIHVLPNIFSMAE
SDLILVLLSLVDMTLVGGLLVMVMFSGYENFVSQLDISENKEKLNWLGKM
DATSLKNKVAASIVAISSIHLLRVFMDAKNVPDNKLMWYVIIHLTFVLSA
FVMGYLDRLTRHNH

Escherichia coli yqhA protein PFAM domain PF03350 sequence:

(SEQ ID NO: 31)
ERFLENAMYASRWLLAPVYFGLSLALVALALKFFQEIIHVLPNIFSMAES
DLILVLLSLVDMTLVGGLLVMVMFSGYENFVSQLDISENKEKLNWLGKMD
ATSLKNKVAASIVAISSIHLLRVFMDAKNVPDNKLMWYVIIHLTFVLSAF

Another non-limiting example of a bacterial protein in the UPF0114 family is the WP_021087398.1 protein in Campylobacter concisus. The UPF0114 PFAM domain PF03350 is shown underneath.

(SEQ ID NO: 22)
MRKIFERILLASNSFTLFPVVFGLLGAIVLFIIASYDVGKVLLEVYKYFF
AADFHVENFHSEVVGEIVGAIDLYLMALVLYIFSFGIYELFISEITQLKQ
SKQSKVLEVHSLDELKDKLGKVIVMVLIVNFFQRVLHANFTTPLEMAYLA
ASILALCLGLYFLHKGDH

Campylobacter concisus WP_021087398.1 protein PFAM domain PF03350 sequence:

(SEQ ID NO: 32)
KIFERILLASNSFTLFPVVFGLLGAIVLFIIASYDVGKVLLEVYKYFFAA
DFHVENFHSEVVGEIVGAIDLYLMALVLYIFSFGIYELFISEITQLKQSK
QSKVLEVHSLDELKDKLGKVIVMVLIVNFFQRVLHANFTTPLEMAYLAAS
ILALCLGLYFLHKGD

Another non-limiting example of a bacterial protein in the UPF0114 family is the OUV44343.1 protein in Rhodobacteraceae bacterium TMED111. The UPF0114 PFAM domain PF03350 is shown underneath.

(SEQ ID NO: 23)
MGFIERIGEKILWNSRFIVILAVIFSIIASISLFIIGSYEIIYSLVYENP
IWSEKYKHNHAQILYKIISAVDLYLIGVVLMIFGFGIYELFISKIDIARK
NPSITILEIENLDELKNKIVKVIVMVLIVSFFERILKNSDAFTSSLNLLY
FAISIFAISFSIYYINKNKN

Rhodobacteraceae bacterium TMED111 PFAM domain PF03350 sequence:

(SEQ ID NO: 33)
ERIGEKILWNSRFIVILAVIFSIIASISLFIIGSYEIIYSLVYENPIWSE
KYKHNHAQILYKIISAVDLYLIGVVLMIFGFGIYELFISKIDIARKNPSI
TILEIENLDELKNKIVKVIVMVLIVSFFERILKNSDAFTSSLNLLYFAIS
IFAISFSIYYIN

A non-limiting example of a green algal protein in the UPF0114 family is the 108867 protein in Micromonas pusilla. The UPF0114 PFAM domain PF03350 is shown underneath.

(SEQ ID NO: 24)
MSSSGVLSLSASARVAPRATSVRRARAPVRATQLARSRADTAAWGKKFMS
VERGSRAVGVRSLVEAANTEPGASYDDGDDHVDTTYDAEDLAHPDVAMMK
ASREVRKPFREFSLIEKVEYVFVRFTLISACIFVLLGVLASLLLSALLFS
MGMKEVLFDAVQAWAGYSPVGLVSSAVGALDRFLLGMVCLVFGLGSFELF
LARSNRAGQVRDRRLKKLAWLKVSSIDDLEQKVGEIIVAVMVVNLLEMSL
HMTYAAPLDLVWAALAAVMSAGALALLHYAAGHGDHNHKDKGGHDSGAGL
LH

Micromonas pusilla 108867 PFAM domain PF03350 sequence:

(SEQ ID NO: 34)
TLISACIFVLLGVLASLLLSALLFSMGMKEVLFDAVQAWAGYSPVGLVSS
AVGALDRFLLGMVCLVFGLGSFELFLARSNRAGQVRDRRLKKLAWLKVSS
IDDLEQKVGEIIVAVMVVNLLEMSLHMTYAAPLDLVWAALAAVMSAGALA
LL

Another non-limiting example of a green algal protein in the UPF0114 family is the GAQ84557.1 protein in Klebsormidium nitens. The UPF0114 PFAM domain PF03350 is shown underneath.

(SEQ ID NO: 25)
MSKDGVAAIDVMMPDGASEDYPITLEEADASDGEWTRRKRHVKRLKKVES
TIERVIFDCRFFALMGVVGSLIGSFLCFVKGCFYVYKAIIAAAFDVTHGL
NSYKVVLKLIEALDTYLVATVMLIFGMGLYELFVNELEAVATTDSVVGCK
SNLFGLFRLRERPKWLQINGLDALKEKLGHVIVMILLVGMFEKSKKVPIR
NGVDLVCVATSVLLCAGSLYLLSQLSKNGNGH

Klebsormidium nitens GAQ84557.1 protein PFAM domain PF03350 sequence:

(SEQ ID NO: 35)
ESTIERVIEDCRFFALMGVVGSLIGSFLCFVKGCFYVYKAIIAAAFDVTH
GLNSYKVVLKLIEALDTYLVATVMLIFGMGLYELFVNELEAVATTDSVVG
CKSNLFGLFRLRERPKWLQINGLDALKEKLGHVIVMILLVGMFEKSKKVP
IRNGVDLVCVATSVLLCAGSLYLL

A non-limiting example of a plant protein in the UPF0114 family is the AT5G13720.1 protein in Arabidopsis thaliana. The UPF0114 PFAM domain PF03350 is shown underneath.

(SEQ ID NO: 26)
MALSSLISATPLSLSVPRYLVLPTRRRFHLPLATLDSSPPESSASSSIPT
SIPVNGNTLPSSYGTRKDDSPFAQFFRSTESNVERIIFDFRFLALLAVGG
SLAGSLLCFLNGCVYIVEAYKVYWTNCSKGIHTGQMVLRLVEAIDVYLAG
TVMLIFSMGLYGLFISHSPHDVPPESDRALRSSSLFGMFAMKERPKWMKI
SSLDELKTKVGHVIVMILLVKMFERSKMVTIATGLDLLSYSVCIFLSSAS
LYILHNLHKGET

Arabidopsis thaliana AT5G13720.1 protein PFAM domain PF03350 sequence:

(SEQ ID NO: 36)
SNVERIIFDFRFLALLAVGGSLAGSLLCFLNGCVYIVEAYKVYWTNCSKG
IHTGQMVLRLVEAIDVYLAGTVMLIFSMGLYGLFISHSPHDVPPESDRAL
RSSSLFGMFAMKERPKWMKISSLDELKTKVGHVIVMILLVKMFERSKMVT
IATGLDLLSYSVCIFLSSASLYIL

Another non-limiting example of a plant protein in the UPF0114 family is the LOC_Os03g52910.1 protein in Oryza sativa. The UPF0114 PFAM domain PF03350 is shown underneath.

(SEQ ID NO: 27)
MAAAAAGGGGGGGGSGRLLRGATAKAFHGDGSSHHRMMPSSSSSVAAGGG
GGVAGPCRIPSLKFPSLWESKRQGGGVGSRAAERKAALIALGAAGVTALE
RERGGGVVLLPEEARRGADLLLPLAYEVARRLVLRQLGGATRPTQQCWSK
IAEATIHQGVVRCQSFTLIGVAGSLVGSVPCFLEGCGAVVRSFFVQFRAL
TQTIDQAEIIKLLIEAIDMFLIGTALLTFGMGMYIMFYGSRSIQNPGMQG
DNSHLGSFNLKKLKEGARIQSITQAKTRIGHAILLLLQAGVLEKFKSVPL
VTGIDMACFAGAVLASSAGVFLLSKLSTTAAQAQRQPRKRTAFA

Oryza sativa LOC 0s03g52910.1 protein PFAM domain PF03350 sequence:

(SEQ ID NO: 37)
ATIHQGVVRCQSFTLIGVAGSLVGSVPCFLEGCGAVVRSFFVQFRALTQT
IDQAEIIKLLIEAIDMFLIGTALLTFGMGMYIMFYGSRSIQNPGMQGDNS
HLGSFNLKKLKEGARIQSITQAKTRIGHAILLLLQAGVLEKFKSVPLVTG
IDMACFAGAVLASSAGVFLLS

As noted above, UPF0114 family proteins for use in the present invention are capable of transporting carboxylates/carboxylic acids (e.g. monocarboxylates/monocarboxylic acids, and/or dicarboxylates/dicarboxylic acids, and/or tricarboxylates/tricarboxylic acids) across biological membranes (e.g. those of organelles and/or the cytoplasmic membrane i.e. the cell membrane surrounding the cytoplasm). The proteins may thus be capable of exporting the carboxylates/carboxylic acids from cell organelles (e.g. chloroplasts, mitochondria) and/or from cells into the external environment. In some embodiments, the UPF0114 family proteins are capable of bidirectional transport of the same or different molecules into and out of cell organelles and/or cells. Additionally or alternatively, the UPF0114 family proteins may be capable of importing and/or exporting molecules (e.g. into and/or out of a cell organelle; into and/or out of a cell) against a concentration gradient, wherein the amount or concentration of the molecule in proximity to a first side of the membrane is below that of the opposing side of the membrane to which the molecule is being transported.

A non-limiting example of a bacterial member of the UPF0114 protein family is the Escherichia coli gene yqhA (UniProt ID P67244, SEQ ID NO: 1).

A non-limiting example of a plant member of the UPF0114 protein family is the (C₃ photosynthetic plant) Arabidopsis thaliana gene AT4G19390 (amino acid sequence: SEQ ID NO: 2). A second non-limiting example of a plant member of the UPF0114 protein family is the (C₄ photosynthetic plant) Setaria italica Si007164m (also known as Seita.4G275500) (amino acid sequence: SEQ ID NO: 3). A third non-limiting example of a plant member of the UPF0114 protein family is the (C₄ photosynthetic plant) Setaria viridis Sevir.4G287300 gene (amino acid sequence: SEQ ID NO: 6). A fourth non-limiting example of a plant member of the UPF0114 protein family is the (C₄ photosynthetic plant) Zea mays GRMZM2G179292 gene (amino acid sequence: SEQ ID NO: 9). A fifth non-limiting example of a plant member of the UPF0114 protein family is the (C₄ photosynthetic plant) Zea mays GRMZM2G133400 gene (amino acid sequence: SEQ ID NO: 10). A sixth non-limiting example of a plant member of the UPF0114 protein family is the (C₄ photosynthetic plant) Zea mays GRMZM2G327686 gene (amino acid sequence: SEQ ID NO: 11). In some embodiments, the UPF0114 protein may be classified as an Embryophyta, Klebsormidiophyceae, Chlorophyta, Viridae, Bacteria, or Archaea protein.

The present invention encompasses homologs, analogs, orthologs and paralogs of the specific UPF0114 proteins and protein sequences provided herein. In view of the high level of evolutionary conservation evident among, for example, viral, bacterial, archaeal, algal, and plant UPF0114 family proteins, the skilled person can identify such homologs, analogs, orthologs and paralogs using routine methods without inventive effort. Numerous publicly accessible online tools are available to the skilled person which can be used to find nucleotide and protein sequences similar to a UPF0114 protein or nucleotide sequence of interest.

Methods for assessing the level of homology and identity between sequences are well known in the art. The percentage of sequence identity between two sequences may, for example, be calculated using a mathematical algorithm. A non-limiting example of a suitable mathematical algorithm is described in the publication of Karlin and colleagues (1993, PNAS USA, 90:5873-5877). This algorithm is integrated in the BLAST (Basic Local Alignment Search Tool) family of programs (see also Altschul et al. (1990), J. Mol. Biol. 215, 403-410 or Altschul et al. (1997), Nucleic Acids Res, 25:3389-3402) accessible via the National Center for Biotechnology Information (NCBI) website homepage (https://www.ncbi.nlm.nih.gov). The BLAST program is freely accessible at https://blast.ncbi.nlm.nih.gov/Blast.cgi. Other non-limiting examples include the HMMER (http://hmmer.org/), (Clustal (http://www.clustal.org/) and FASTA (Pearson (1990), Methods Enzymol. 83, 63-98; Pearson and Lipman (1988), Proc. Natl. Acad. Sci. U. S. A 85, 2444-2448.) programs. These and other programs can be used to identify sequences which are at least to some level identical to a given input sequence. Additionally or alternatively, programs available in the Wisconsin Sequence Analysis Package, version 9.1 (Devereux et al. 1984, Nucleic Acids Res., 387-395), for example the programs GAP and BESTFIT, may be used to determine the percentage of sequence identity between two polypeptide sequences. BESTFIT uses the local homology algorithm of Smith and Waterman (1981, J. Mol. Biol. 147, 195-197) and identifies the best single region of similarity between two sequences. Where reference herein is made to an amino acid sequence sharing a specified percentage of sequence identity to a reference amino acid sequence, the difference/s between the sequences may arise partially or completely from amino acid substitution/s. In such cases, the sequence identified with the amino acid substitution/s may substantially or completely retain the same biological activity of the reference sequence.

### Sequence Modifications

UPF0114 protein family sequences of the present invention may be modified to enhance expression in a recombinant cell. Many publicly available online tools exist to enable the skilled artisan to optimise a nucleotide or protein sequence for use in the present invention (see, for example, http://genomes.urv.es/OPTIMIZER).

For example, the sequence may be modified by codon optimisation. As known to those of skill in the art, organisms differ in their tendency to use specific codons over others to encode the same amino acid. Codon optimisation may thus be employed to enhance expression of UPF0114 protein sequences in specific cell types.

Additionally or alternatively, nucleotide sequences encoding UPF0114 family proteins of the present invention may be modified by the removal of one or more introns.

Additionally or alternatively, nucleotide sequences encoding UPF0114 family proteins of the present invention may be modified by operably linking them to regulatory sequences (e.g. promoters, enhancers and the like) to manipulate the level at which they are transcribed.

Additionally or alternatively, UPF0114 protein family sequences of the present invention may be manipulated to direct the movement of the proteins to specific internal cellular locations (e.g. the envelope membranes of organelles such as a chloroplast or mitochondria) or to the cytoplasmic membrane itself (i.e. the cell membrane surrounding the cytoplasm). For example, the sequences may be operably linked to a signal peptide or targeting peptide sequence, or alternatively have an existing signal peptide sequence removed.

Additionally or alternatively, UPF0114 protein family sequences of the present invention may be manipulated to facilitate detection and/or isolation by way of incorporating tag sequences or the like.

The skilled addressee will recognise that the examples of sequence modifications above are non-limiting, with many other known sequence modifications available that could be used as a matter of routine. The present invention contemplates any and all modifications of this nature.

### Carboxylates

UPF0114 family proteins of the present invention are used to transport carboxylates, and in particular any one or more of monocarboxylates/monocarboxylic acids, and/or dicarboxylates/dicarboxylic acids, and/or tricarboxylates/tricarboxylic acids.

In some embodiments of the present invention, the carboxylates/carboxylic acids may comprise or consist of monocarboxylates/monocarboxylic acids. For example, the monocarboxylates/monocarboxylic acids may comprise or consist of pyruvate/pyruvic acid. Additionally or alternatively, the monocarboxylates/monocarboxylic acids may comprise or consist of any one or more of: lactate/lactic acid, glycerate/glyceric acid, acetate/acetic acid, branched-chain oxo acids, acetoacetate, betα-hydroxybutyrate.

In some embodiments of the present invention, the carboxylates/carboxylic acids may comprise or consist of dicarboxylates/dicarboxylic acids. For example, the dicarboxylates/dicarboxylic acids may comprise or consist of any one or more of: succinate/succinic acid, malate/malic acid, fumarate/fumaric acid, α-ketoglutarate/α-ketoglutaric acid, aspartate/aspartic acid, glutamate/glutamic acid.

In other embodiments of the present invention, the carboxylates/carboxylic acids may comprise or consist of tricarboxylates/tricarboxylic acids. For example, the tricarboxylates/tricarboxylic acids may comprise or consist of any one or more of: citrate/citric acid, isocitrate/isocitric acid, aconitate/aconitic acid, propane-1,2,3-tricarboxylic acid, trimesic acid.

In still other embodiments of the present invention, the carboxylates/carboxylic acids may be phosphorylated. Accordingly, the UPF0114 family proteins of the present invention may be used to transport any one or more of: phosphorylated monocarboxylates/monocarboxylic acids, phosphorylated dicarboxylates/dicarboxylic acids, phosphorylated tricarboxylates/tricarboxylic acids. Non-limiting examples of phosphorylated carboxylic acids that may be transported by the UPF0114 family proteins include glycerate-3-phosphate/3-phosphoglyceric acid and phosphoenolpyruvate/phosphoenolpyruvic acid.

As noted above, UPF0114 family proteins of the present invention may be capable of bidirectional movement of carboxylates/carboxylic acids across biological membranes. In some embodiments, the UPF0114 family proteins may be capable of the uptake of malate and the export of more pyruvate. Additionally or alternatively, the UPF0114 family proteins may be capable of exporting any one of more of lactate, succinate, malate, fumarate, glycerate, α-ketoglutarate, aspartate, aconitate, citrate, branched-chain oxo acids, acetoacetate, betα-hydroxybutyrate from an organelle (e.g. a chloroplast), a cell (e.g. a bacterial, plant or algal cell). This transport may occur with or against a concentration gradient.

### Recombinant Cells

The present invention provides recombinant cells expressing UPF0114 family proteins. The UPF0114 family protein may be encoded by a recombinant nucleic acid sequence (e.g. recombinant DNA, recombinant RNA, and the like) introduced into the base cell.

For example, a recombinant nucleic acid sequence encoding a UPF0114 family protein may be transiently introduced into the cell. This may result in transient expression of the UPF0114 family proteins for a finite period (e.g. 1, 2, 3, 4, 5, 7, 8, 9, or 10 days). Methods for achieving transient expression of recombinant nucleic acids in host cells are well known in the art. In some embodiments, transient expression may be characterised by a lack of replication of the recombinant nucleic acid sequence when the host cell replicates. In some embodiments, transient expression may be characterised by an absence of integration of the recombinant nucleic acid sequence into the genome of the host cell.

Additionally or alternatively, a recombinant nucleic acid sequence encoding a UPF0114 family protein may be stably introduced into the cell. Recombinant nucleic acid sequences that have been stably introduced into the cell will generally be replicated when the host cell replicates. In some embodiments, stable expression may be characterised by integration of the recombinant nucleic acid sequence into the genome of the host cell. In some embodiments, stable expression may be characterised by introducing the recombinant nucleic acid sequence into the cell as a component of a vector (e.g. an expression vector). Suitable vectors for this purpose are well known to those of skill in the art and include, without limitation, plasmids, cosmids, yeast vectors, yeast artificial chromosomes, bacterial artificial chromosomes, P1 artificial chromosomes, plant artificial chromosomes, algal artificial chromosomes, modified viruses (e.g. modified adenoviruses, retroviruses or phages), and mobile genetic elements (e.g. transposons).

Techniques for producing recombinant nucleic acids (e.g. recombinant DNA, recombinant RNA, and the like) including those provided in the form of a vector, are well known to those skilled in the art, as are techniques for the introduction of recombinant nucleic acids into cells (e.g. electroporation, microinjection, biolistic delivery systems, calcium phosphate co-precipitation, cationic lipid-based transfection reagents, diethylaminoethyl-dextran). General guidance on suitable methods can be found, for example, in standard texts such as Green and Joseph. (2012), Molecular cloning: a laboratory manual, fourth edition. Cold Spring Harbor, N.Y.: Cold Spring Harbor Laboratory Press; Ausubel et al. (1987-2016). Current Protocols in Molecular Biology. New York, N.Y., John Wiley & Sons; and ‘Cloning a Specific Gene.’ in Griffiths et al. 1999 Modern Genetic Analysis. New York: W.H. Freeman.

The recombinant cell may be any suitable type including, but not limited to, prokaryotic, eukaryotic, archaeal, plant, algal, bacterial, yeast, fungal, animal, mammalian, or synthetic cells.

In some embodiments, the host cell may be bacterial cell such as, for example, Escherichia coli or Agrobacterium tumefaciens. The bacterial cell may be autotrophic (e.g. a cyanobacterium).

In other embodiments, the host cell may be a plant cell (e.g. a C₃ photosynthetic plant cell, such as a C₃ plant vascular sheath cell, a C₃ plant bundle sheath cell, a C₃ plant mestome sheath cell, or a C₃ plant mesophyll cell; a C₄ photosynthetic plant cell such as a C₄ plant vascular sheath cell, a C₄ plant bundle sheath cell, a C₄ plant mestome sheath cell or a C₄ plant mesophyll cell; or a CAM photosynthetic plant cell, such as a CAM plant vascular sheath cell, a CAM plant bundle sheath cell, a CAM plant mestome sheath cell or a CAM plant mesophyll cell).

In still other embodiments, the host cell may be yeast such as, for example, Saccharomyces cerevisiae, Pichia pastoris, Pichia methanolica and Hansenula polymorpha.

The recombinant cells expressing carboxylates/carboxylic acids of the present invention may also be engineered to produce carboxylates/carboxylic acids. For example, the recombinant cells may further produce any one or more of monocarboxylates/monocarboxylic acids, and/or dicarboxylates/dicarboxylic acids, and/or tricarboxylates/tricarboxylic acids. Additionally or alternatively, the recombinant cells may be engineered to produce or overexpress enzyme/s and/or regulatory protein/s of biochemical pathway/s for production of the carboxylates/carboxylic acids (e.g. for production of monocarboxylates/monocarboxylic acids, and/or dicarboxylates/dicarboxylic acids, and/or tricarboxylates/tricarboxylic acids).

Production of the carboxylates/carboxylic acids and/or enzyme/s and/or regulatory protein/s in the recombinant cells can be achieved, for example, using the same materials and techniques as described above in relation to the overexpression of the UPF0114 family proteins.

Non-limiting examples of monocarboxylates/monocarboxylic acids that may be produced by the recombinant cells include any one more of: pyruvate/pyruvic acid, lactate/lactic acid, glycerate/glyceric acid, acetate/acetic acid, branched-chain oxo acids, acetoacetate, betα-hydroxybutyrate.

Non-limiting examples of dicarboxylates/dicarboxylic acids that may be produced by the recombinant cells include any one or more of: succinate/succinic acid, malate/malic acid, fumarate/fumaric acid, α-ketoglutarate/α-ketoglutaric acid, aspartate/aspartic acid, glutamate/glutamic acid.

A non-limiting example of a tricarboxylates/tricarboxylic acid that may be produced by the recombinant cells include any one or more of: citrate/citric acid, isocitrate/isocitric acid, aconitate/aconitic acid, propane-1,2,3-tricarboxylic acid, trimesic acid.

The carboxylates/carboxylic acids produced in the recombinant cells may be phosphorylated (e.g. phosphorylated monocarboxylates/monocarboxylic acids, and/or phosphorylated dicarboxylates/dicarboxylic acids, and/or phosphorylated tricarboxylates/tricarboxylic acids). Non-limiting examples include glycerate-3-phosphate/3-phosphoglyceric acid and phosphoenolpyruvate/phosphoenolpyruvic acid.

The enzyme/s and/or regulatory protein/s of biochemical pathway/s for production of the carboxylates/carboxylic acids that may be produced in the recombinant cell include, for example, any one or more of: pyruvate carboxylase, pyruvate synthase, pyruvate dehydrogenase, pyruvate kinase, citrate synthase, aconitase, isocitrate dehydrogenase, α-ketoglutarate dehydrogenase, Succinyl-CoA synthase, succinic dehydrogenase, fumarase, malate dehydrogenase, malic enzyme, phosphoenolpyruvate carboxykinase, malate quinone-oxidoreductase, glutamate dehydrogenase, lactate dehydrogenase, isocitrate lyase, malate synthase.

### Transgenic Plants

Recombinant plants cells of the present invention may be used to generate transgenic plants. In some embodiments of the present invention, the transgenic plants have an increased rate of photosynthesis relative to the unmodified plant line.

By way of non-limiting example, a C₃ photosynthetic plant cell (e.g. a C₃ plant vascular sheath cell, a C₃ plant mestome sheath cell, a C₃ plant mesophyll cell, or a C₃ plant bundle sheath cell) may be engineered to express or overexpress a UPF0114 family protein capable of importing and/or exporting carboxylates/carboxylic acids (e.g. monocarboxylates/monocarboxylic acids, and/or dicarboxylates/dicarboxylic acids, and/or tricarboxylates/tricarboxylic acids) across membrane/s of the cell (e.g. those of organelles such as chloroplasts and/or mitochondria, and/or the cytoplasmic membrane). The UPF0114 family protein may, for example, be a UPF0114 protein from a C₃ plant, a C₄ plant, a CAM plant, an alga, a virus, a bacterium or an archaeon.

In some embodiments, the UPF0114 family protein may be capable of importing malate into any cell type or subcellular organelle within a C₃ plant including but not limited to a C₃ plant mesophyll cell, a C₃ plant bundle sheath cell, a C₃ plant mesophyll cell chloroplast, a C₃ plant bundle sheath cell chloroplast, a C₃ plant mesophyll cell mitochondrion, a C₃ plant bundle sheath cell mitochondrion. Additionally or alternatively, the UPF0114 family protein may be capable of exporting pyruvate from any cell type or subcellular organelle within a C₃ plant including but not limited to: a C₃ plant mesophyll cell, a C₃ plant bundle sheath cell, a C₃ plant mesophyll chloroplast, a C₃ plant bundle sheath cell chloroplast.

By way of further non-limiting example, a C₄ photosynthetic plant cell (e.g. a C₄ plant vascular sheath cell, a C₄ plant bundle sheath cell, a C₄ plant mestome sheath cell or a C₄ plant mesophyll cell) may be engineered to express or overexpress a UPF0114 family protein capable of importing and/or exporting carboxylates/carboxylic acids (e.g. monocarboxylates/monocarboxylic acids, and/or dicarboxylates/dicarboxylic acids, and/or tricarboxylates/tricarboxylic acids) across membrane/s of the cell (e.g. those of organelles such as chloroplasts and/or mitochondria, and/or the cytoplasmic membrane). The UPF0114 family protein may, for example, be a UPF0114 protein from a C₃ plant, a C₄ plant, a CAM plant, an alga, a virus, a bacterium or an archaeon.

In some embodiments, the UPF0114 family protein may be capable of importing malate into any cell type or subcellular organelle within a C₄ plant including but not limited to: a C₄ plant mesophyll cell, a C₄ plant bundle sheath cell, a C₄ plant mesophyll cell chloroplast, a C₄ plant bundle sheath cell chloroplast, a C₄ plant mesophyll cell mitochondrion, a C₄ plant bundle sheath cell mitochondrion. Additionally or alternatively, the UPF0114 family protein may be capable of exporting pyruvate from any one or more of: a C₄ plant mesophyll cell, a C₄ plant bundle sheath cell, a C₄ plant mesophyll chloroplast, a C₄ plant bundle sheath cell chloroplast.

By way of further non-limiting example, a plant cell that conducts crassulacean acid metabolism (CAM) (e.g. a CAM plant vascular sheath cell, a CAM plant bundle sheath cell, a CAM plant mestome sheath cell, a CAM plant mesophyll cell, or a CAM plant bundle sheath cell) may be engineered to express or overexpress a UPF0114 family protein capable of importing and/or exporting carboxylates/carboxylic acids (e.g. monocarboxylates/monocarboxylic acids, and/or dicarboxylates/dicarboxylic acids, and/or tricarboxylates/tricarboxylic acids) across membrane/s of the cell (e.g. those of organelles such as chloroplasts and/or mitochondria, and/or the cytoplasmic membrane). The UPF0114 family protein may, for example, be a UPF0114 protein from a C₃ plant, a C₄ plant, a CAM plant, an alga, a virus, a bacterium or an archaeon.

In some embodiments, the UPF0114 family protein may be capable of importing malate into any cell type or subcellular organelle within a CAM plant including but not limited to: a CAM plant mesophyll cell, a CAM plant bundle sheath cell, a CAM plant mesophyll cell chloroplast, a CAM plant bundle sheath cell chloroplast, a CAM plant mesophyll cell mitochondrion, a CAM plant bundle sheath cell mitochondrion. Additionally or alternatively, the UPF0114 family protein may be capable of exporting pyruvate from any one or more of: a CAM plant mesophyll cell, a CAM plant bundle sheath cell, a CAM plant mesophyll chloroplast, a CAM plant bundle sheath cell chloroplast.

Methods for producing transgenic plants are well known to persons skilled in the art (see, for example, Gamborg and Phillips, 1995, Plant cell, tissue and organ culture: fundamental methods. Springer, Berlin; Low et al. 2018, ‘Transgenic Plants: Gene Constructs, Vector and Transformation Method’ in New Visions in Plant Science, Çelik (Ed), IntechOpen; Transgenic Crop Plants, Volume 1. Principles and Development, 2010, Kole, Michler, Abbott, Hall, (Eds.)).

In some embodiments, the transgenic plants may be monocotyledonous. In other embodiments, the transgenic plants may be dicotyledonous. In still other embodiments, the transgenic plants may be a genus Oryza plant such as, for example, a rice plant (e.g. a Oryza sativa plant or a Oryza glaberrima plant).

In some embodiments, the transgenic plant may be soy (Glycine max), cotton (Gossypium hirsutum), oilseed rape/Cannola (B. napus subsp. Napus), potato (Solanum tuberosum), tomato (Solanum lycopersicum), cassava (Manihot esculenta), maize (Zea mays), sorghum (Sorghum bicolor), sugar cane (Saccharum officinarum), foxtail millet (Setaria italica), proso millet (Panicum miliaceum), mischanthus (Miscanthus giganteus), wheat (Triticum aestivum), barley (Hordeum vulgare), pigeon pea (Cajanus cajan), cowpea (Vigna unguiculata), pea (Pisum sativum), cannabis (Cannabis sativa), sugar beet (Beta vulgaris), oat (Avena sativa), rye (Secale cereal), peanut (Arachis hypogaea), sunflower (Helianthus annuus), flax (Linum spp.), beans (Phaseolus vulgaris), lima bean (Phaseolus lunatus), mung bean (Phaseolus mung), adzuki bean (Phaseolus angularis), Chickpea (Cicer arietinum), tobacco (Nicotiana tabacum), buckwheat (Fagopyrum esculentum), oil palm (Elaeis guineensis), or rubber (Hevea brasiliensis).

Also provided are seeds obtained from the transgenic plants of the present invention.

### Methods of Use

Provided herein are methods for exploiting the recombinant cells of the present invention.

Without limitation, the recombinant cells may be used in metabolite production given that they provide a means of exporting carboxylates/carboxylic acids with or against concentration gradients. For example, the recombinant cells of the present invention can be used in the commercial production of carboxylates such as pyruvate or succinate, which may in turn be used as building blocks for a large range of complex chemicals, non-limiting examples of which include polymers, solvents and pharmaceuticals. In some embodiments, biological production of these metabolites may occur by fermentation from cheaper sugars. The microorganisms currently used for bioproduction of carboxylates either naturally, or have been engineered to, accumulate high concentrations of carboxylates within the cell. A large component of the cost of biological production of these metabolites is attributable to the process of extracting the metabolites from the cells and subsequently separating them from other cellular contaminants. Thus, the recombinant cells and methods of the present invention may provide a substantial reduction in the cost of carboxylate production by specifically exporting these metabolites from cells during the process of fermentation. In other embodiments, carboxylates may be overexpressed in the recombinant cells of the present invention, and similarly exported via UPF0114 family proteins engineered into membrane/s of the cell to facilitate more efficient and simplified collection.

Further methods of the present invention involve the generation of transgenic plants as described above. The transgenic plants will ideally have an increased photosynthetic rate as compared to a corresponding wild-type plant. In some embodiments, the transgenic plants are constructed from C₃ photosynthetic plants to include C₄ photosynthetic traits. In other embodiments, the transgenic plants are constructed from C₃ photosynthetic plants to include crassulacean acid metabolism (CAM) photosynthetic traits. In still other some embodiments, the transgenic plants are constructed from C₄ photosynthetic plants in which photosynthesis has been improved by overexpression of UPF0114 family proteins.

### EXAMPLES

The present invention will now be described with reference to specific Examples, which should not be construed as in any way limiting.

### Example One

### The Gene Family Encodes a Family of Carboxylate and Phosphorylated Carboxylate Transporters

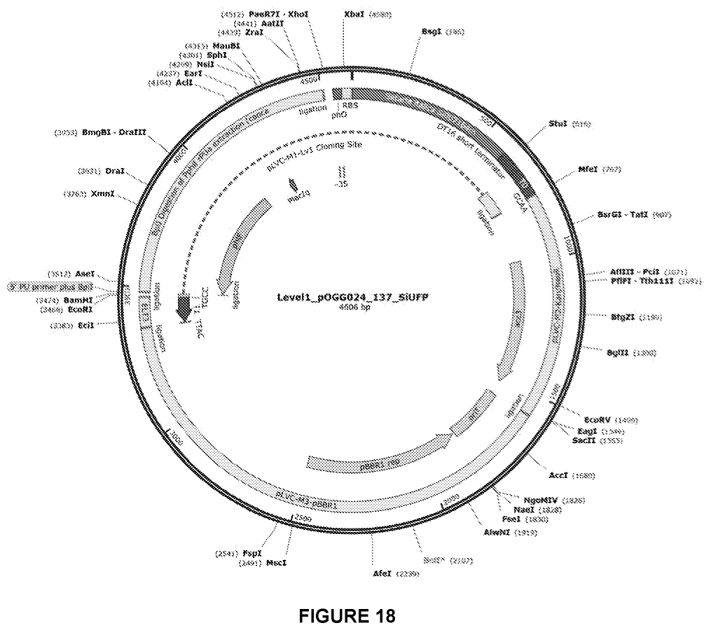

To characterise the transport activities of these representative members of this gene family the genes were cloned into an inducible expression vector (FIG. 18).

In total the transport activities of the proteins encoded by 8 different members of the UPF0114 gene family were subject to experimental interrogation. These comprised 1) The protein encoded by the yqhA gene in Escherichia coli for which the complete amino acid sequence shown in SEQ ID NO: 1. 2) The protein encoded by the AT4G19390 gene in Arabidopsis thaliana for which the complete amino acid sequence shown in SEQ ID NO: 2. 3) The protein encoded by the Sevir.4G287300 gene in Setaria viridis for which the complete amino acid sequence shown in SEQ ID NO: 6. 4) The protein encoded by the GRMZM2G179292 gene in Zea mays for which the complete amino acid sequence shown in SEQ ID NO: 9. 5) The protein encoded by the GRMZM2G133400 gene in Zea mays for which the complete amino acid sequence shown in SEQ ID NO: 10. 6) The protein encoded by the GRMZM2G327686 gene in Zea mays for which the complete amino acid sequence shown in SEQ ID NO: 11. In the case of the Escherichia coli yqhA gene, a nucleotide sequence encoding the complete amino acid sequence shown in SEQ ID NO: 1 was used and this gene was cloned into the inducible expression plasmid to generate plasmid 1.

In the case of the Arabidopsis thaliana, Setaria viridis and Zea mays member of the gene family, the nucleotide sequences corresponding to the protein sequences described above were designed to be codon optimised for expression in E. coli. In addition, the introns present in these genes were removed such that the nucleotide sequence comprised only coding sequence. Furthermore, the chloroplast transit peptides were removed to prevent misfolding or mistargeting of the protein in E. coli. These synthetic nucleotide sequences are shown in SEQS ID NOs: 7, 8, 12, 13 and 14. These genes were individually cloned into the inducible expression plasmid to generate plasmids 2-6.

Independent E. coli cell lines were generated such that each contained one of the inducible plasmids listed above. Specifically, cell line 1 contained plasmid 1, cell line 2 contained plasmid 2, cell line 3 contained plasmid 3, cell line 4 contained plasmid 4, cell line 5 contained plasmid 5, cell line 6 contained plasmid 6.

To characterise the metabolites that were exported by the transporters cell lines 1, 2 and 3 (containing the plasmids expressing yqhA, AT4G19390 and Sevir.4G287300 respectively) were grown in M9 minimal medium supplemented with 22mM glucose as the sole carbon source (henceforth referred to as M9 glucose). No other carbon containing molecules were added to the medium and thus glucose was the sole carbon source available to the cells for growth and respiration.

These three cell lines were pre-grown over night from a cell culture with an optical density measured at a wavelength of 600 nm (0D600) of 0.1 in 50m1 in M9 glucose. The following day, each cell line was subcultured to an OD600 of 0.1 in M9 glucose in two separate flasks. Both flasks were allowed to grow to an OD600 of 0.2 and then expression of the transporter gene was induced in one flask by addition of 50 μM 2,4-diacetylphloroglucinol (DAPG) to the cell culture medium. As DAPG stock solution was dissolved in ethanol, an equivalent volume of ethanol without DAPG was added to the non-induced control flasks. Samples of cell culture were taken from both the induced and non-induced control flasks at time 0 and at three hours following induction of transporter gene expression. The cell culture was spun at 13,000 g for five minutes at 4° C. Following centrifugation, the supernatant was aspirated and the cell pellet discarded. In each case, 20 μl of ice-cold supernatant was subject to metabolite extraction by mixing with 350 μl of CHCl₃/CH₃OH (3:7 v/v) and incubating at −20° C. for two hours with mixing. At two hours, 350 μl of ice-cold water was added to this mixture and allowed to warm up to 4° C. This mixture was centrifuged at 13,000 g for ten minutes at 4° C. After this, the upper aqueous-CH₃OH phase was transferred to a 1.5 ml tube. This remaining CHCl₃ phase was re-extracted with 300 μl of ice-cold water and the upper aqueous-CH₃OH phase was removed as before. The two upper aqueous-CH3OH phases were then combined and dried using a centrifugal vacuum dryer. Samples were analysed by LC-MS/MS with authentic standards for accurate metabolite quantification.

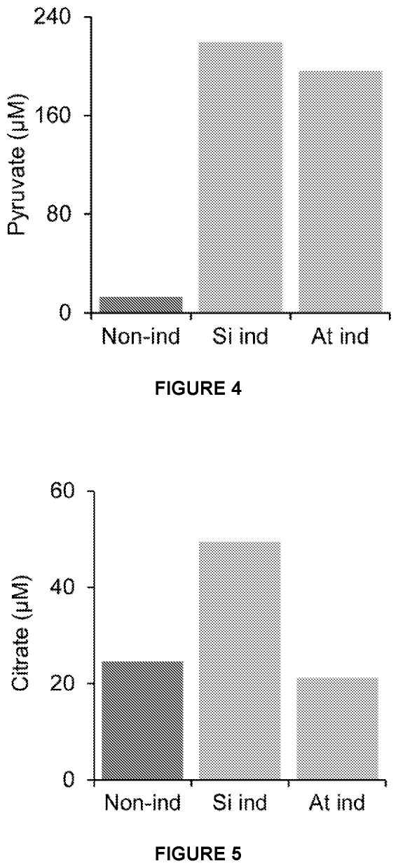

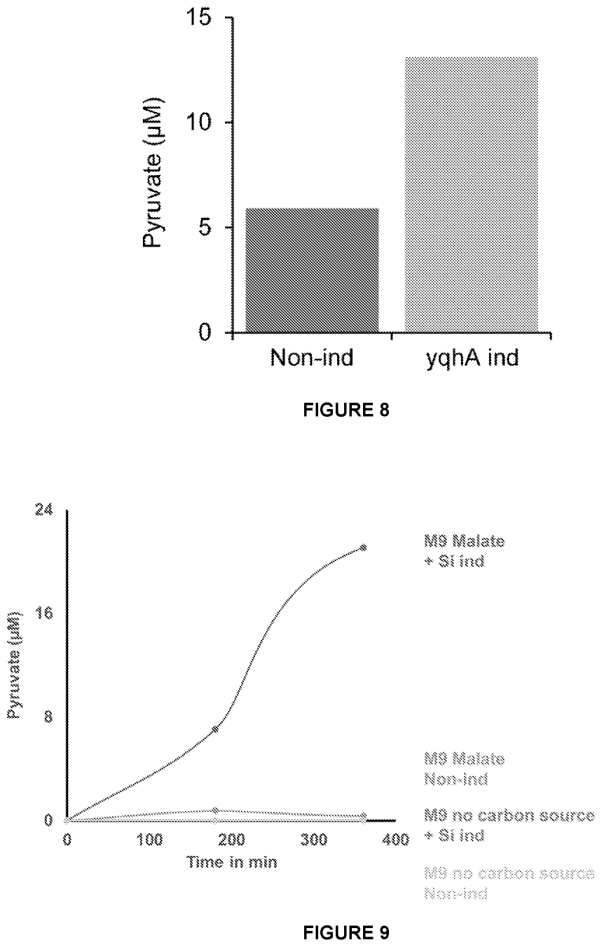

Expression of all three transporters (E. coli yqhA, A. thaliana AT4G19390, and Setaria viridis Sevir.4G287300) resulted in the export of the monocarboxylate/monocarboxylic acid pyruvate to the cell culture medium (FIG. 4 and FIG. 8). Expression of the E. coli gene did not result in any detectable levels of export dicarboxylates/dicarboxylic acids, tricarboxylates/tricarboxylic acids or phosphorylated carboxylates.

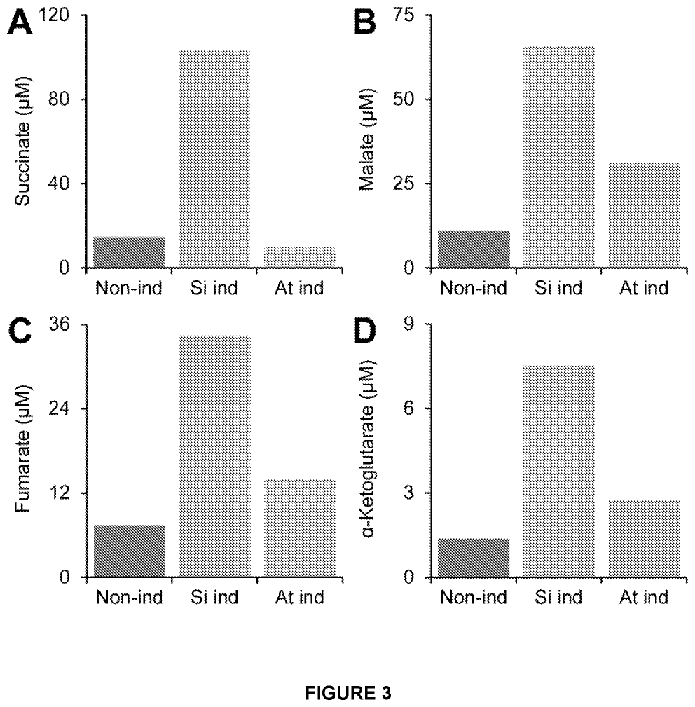

Expression of both of the representative plant members of this gene family resulted in the export of a range of dicarboxylates/dicarboxylic acids (FIG. 3). These include succinate, malate, fumarate, and α-ketoglutarate. Export rates for different dicarboxylates/dicarboxylic acids varied between the two different representative members of the plant gene family tested here. While the Setaria viridis member of the gene family exported all of the listed metabolites, the Arabidopsis thaliana member of the gene family did not export succinate.

Expression of the Setaria viridis member of this gene family resulted in the export of the tricarboxylates/tricarboxylic acid citrate (FIG. 5).

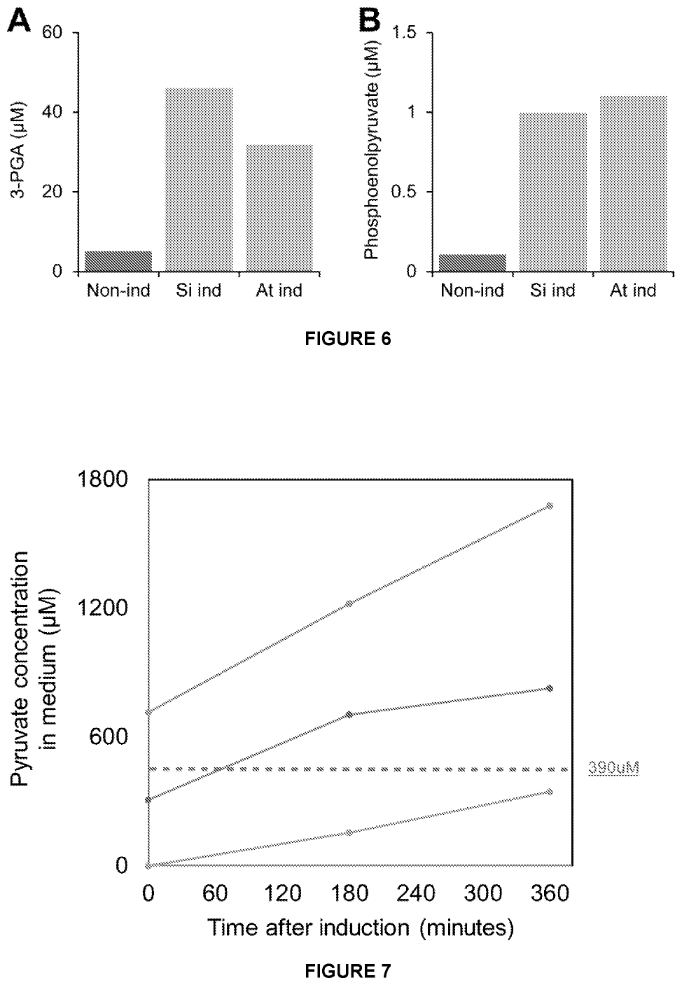

Expression of both of the representative plant members of this gene family resulted in the export of a range of phosphorylated carboxylates (FIG. 6).

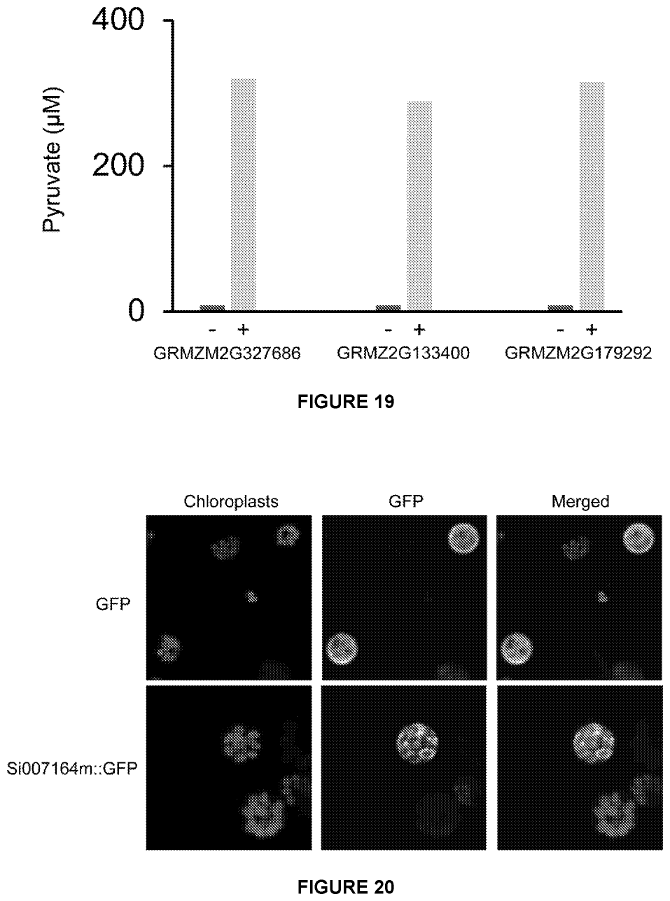

To confirm that all members of the gene family share this transport function the cell lines plasmids 4, 5 and 6 were also subject to analysis. Here these cell lines pre-grown over night from a cell culture with an optical density measured at a wavelength of 600 nm (0D600) of 0.1 in 50m1 in M9 glucose. The following day, each cell line was subcultured to an OD600 of 0.1 in M9 glucose in two separate flasks. Both flasks were allowed to grow to an OD600 of 0.2 and then expression of the transporter gene was induced in one flask by addition of 50 μM 2,4-diacetylphloroglucinol (DAPG) to the cell culture medium. As DAPG stock solution was dissolved in ethanol, an equivalent volume of ethanol without DAPG was added to the non-induced control flasks. Samples of cell culture were taken from both the induced and non-induced control flasks at time 0 and at six hours following induction of transporter gene expression. The cell culture was spun at 13,000 g for five minutes at 4° C. Following centrifugation, the supernatant was aspirated and the cell pellet discarded. The concentration of pyruvate in cell culture supernatants was assessed using a pyruvate oxidase-based enzymatic assay with colorimetric detection (abcam ab65342) according to the manufacturer's instructions. Colorimetric detection was performed using a plate reader (FLUOstar Omega, BMG Labtech), and pyruvate concentration calculated by comparison to the standard curve. In all cases, the expression of the genes encoding different members of the UPF0114 protein family resulted in the export of the monocarboxylate pyruvate. Pyruvate was not exported from non-induced cells (FIG. 19). Thus, given the distribution of the sampled members of the gene family in bacteria and across plants all members of this gene family carry out the same transport reactions.

### Example Two

### The Transporter Can Transport Metabolites Both With and Against a Concentration Gradient

The intracellular concentration of pyruvate in E. coli is 390 μM. To demonstrate that the transporter can export metabolites against a concentration gradient the experiment described in Example one was repeated using the nucleotide sequence of the Sevir.4G287300 gene from Setaria viridis (amino acid sequence shown in SEQ ID NO: 6). This time the M9 glucose growth medium was supplemented with different concentrations of additional pyruvate such that the concentration of pyruvate outside the cell was higher than inside the cell. Initial starting concentrations were chosen to be 0 μM, 300 μM and 700 μM. In all cases, pyruvate was exported from the cells. In the case of both the 300 μM and 700 μM starting concentrations, pyruvate was exported such that pyruvate accumulated to concentrations exceeding the intracellular concentration by three hours (FIG. 7).

Example Three: The transporters facilitate bidirectional transport of metabolites

Under aerobic conditions the dicarboxylate/dicarboxylic acid transporter dctA is solely responsible for uptake of dicarboxylates in E. coli. When the gene encoding dctA is deleted from the E. coli genome, dicarboxylates/dicarboxylic acids can no longer enter the cell and thus E. coli cannot grow on malate as a sole carbon source (FIG. 17). However, uptake of glucose and subsequently growth on glucose as a sole carbon source is not affected (FIG. 17).

The inducible expression plasmid containing the Sevir.4G287300 gene from Setaria viridis was transformed into the dctA knockout line (ΔdctA). ΔdctA lines harbouring the inducible expression plasmid were pre-grown over night from a cell culture with OD600 of 0.1 in 50m1 in M9 glucose. The following day, the cell line was subcultured to an OD600 of 0.2 in M9 glucose in two separate flasks. Expression of the transporter gene was induced in one flask by addition of 50 mM 2,4-diacetylphloroglucinol (DAPG) to the cell culture medium. As DAPG stock solution was dissolved in ethanol, an equivalent volume of ethanol without DAPG was added to the non-induced control flasks. Cell lines were incubated for 2 hours to allow transporter gene expression. Cells were subsequently isolated by centrifugation at 13,000 g for 5 min, washed twice in M9 (+/−DAPG as appropriate) with no carbon source. Cells were then resuspended in M9 malate (+/−DAPG as appropriate) and samples of cell-free supernatant were collected after two and three hours. Pyruvate levels were measured in the supernatant using a colorimetric assay. Pyruvate was readily exported from the cells in the presence of malate, but not in the absence of malate as a carbon source (FIG. 9). As there is no other possible route for malate to enter the cell, and as the transporter is able to export malate from the cell (FIG. 3), the transporter must also therefore also be able to uptake malate from the cell culture medium (FIG. 9).

### Example Four

### In C₃ Plants the Transporter Localises to Chloroplasts

The AT4G19390 gene from Arabidopsis thaliana was tested for subcellular localisation using C-terminal GFP fusions in Arabidopsis thaliana leaf protoplasts. The nucleotide sequence corresponding to the full length amino acid sequence including the predicted chloroplast transit peptide (SEQ ID NO: 2) and with original endogenous codon use, but lacking any introns, was expressed from a constitutive expression vector. The same vector expressing GFP was used as a control.

The Arabidopsis thaliana AT4G19390 gene expressed as a C-terminal GFP fusion in leaf cell protoplasts localised to foci on the periphery in chloroplasts (FIG. 13). GFP on its own localised to the cytosol (FIG. 13).

To further confirm this localisation in C₃ plants a C-terminal GFP fusion of the Seita.4G275500 gene from Setaria italica (SEQ ID NO: 8) was expressed in protoplasts isolated from Oryza sativa (rice) sheath tissue (FIG. 20). The nucleotide sequence corresponding to the full length amino acid sequence, including the predicted chloroplast transit peptide was codon optimised for expression in rice. Following codon optimisation, the first intron from the Sevir.4G287300 gene from Setaria viridis was added to prevent expression in E. coli. The C-terminal translational fusion with GFP was placed under control of the Zea mays Ubiquitin promoter and assembled into a binary vector pL1V-F1-47732. A construct containing the GFP coding sequence driven by the Z. mays Ubiquitin promoter was used as a positive control for cytosolic protein localisation. The protein encoded by the Setaria italica gene fused to GFP localised to the periphery of the chloroplast (FIG. 20) consistent with its predicted localisation of the chloroplast envelope membrane and consistent with the localisation observed in Arabidopsis thaliana protoplasts.

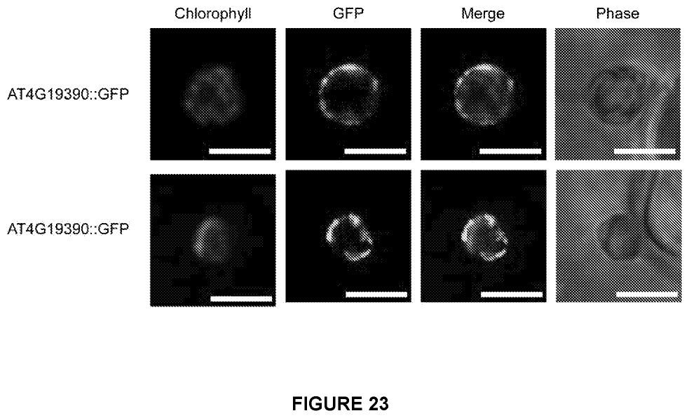

To further confirm this localisation in C₃ plants a C-terminal GFP fusion of the AT4G19390 gene from Arabidopsis thaliana (SEQ ID NO: 2) was expressed in intact plant leaves from Nicotiana benthamiana (FIG. 23). The nucleotide sequence corresponding to the full length amino acid sequence, including the predicted chloroplast transit peptide but lacking any introns was cloned into an expressipon vector for expression in Nicotiana benthamiana. The vector was transfected into Agrobacterium and the transfect agrobacterium infiltrated into the leaves of Nicotiana benthamiana plants. The AT4G19390::GFP protein localised to the periphery of the chloroplast consistent with the localisation observed in Arabidopsis thaliana, Oryza sativa and Setaria italica. Thus, either the C₃ or the C₄ variants of the protein can be expressed in C₃ or C₄ plants and localise to the correct subcellular location.

### Example Five

### In C₄ Plants the Transporter Can Localise to the Chloroplast and to the Plasma Membrane

The Setaria italica member of this gene family was tested for subcellular localisation using C-terminal GFP fusions in Setaria viridis leaf protoplasts. The nucleotide sequence corresponding to the full length amino acid sequence including the predicted chloroplast transit peptide (SEQ ID NO: 3) and with original endogenous codon use, but lacking any introns, was expressed from a constitutive expression vector. The same vector expressing GFP was used as a control.

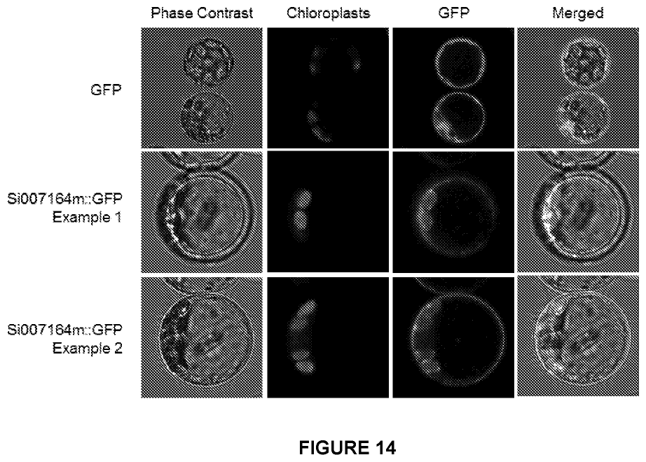

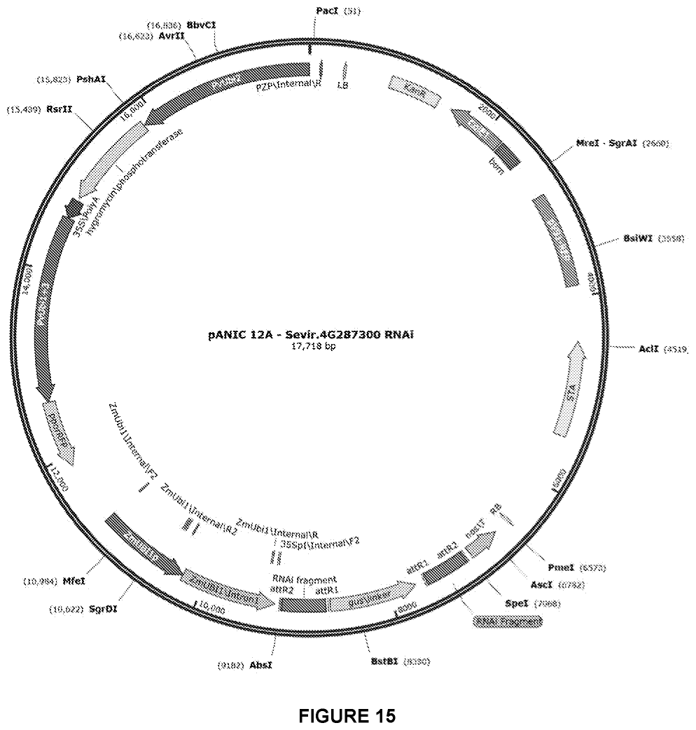

The Setaria italica gene expressed as a C-terminal GFP fusion in leaf cell protoplasts localised to foci in chloroplasts (FIG. 14). There was also some localisation to the plasma membrane (FIG. 14). GFP on its own localised to the cytosol (FIG. 14).

Example Six: RNAi knockdown of the transporter disrupts C₄ photosynthesis

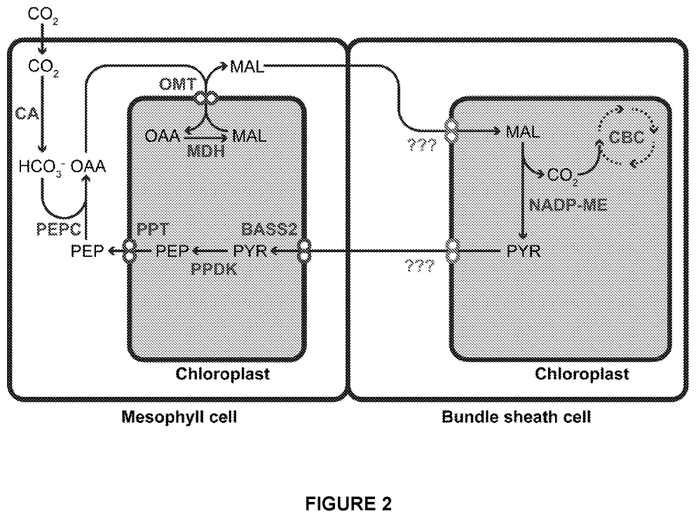

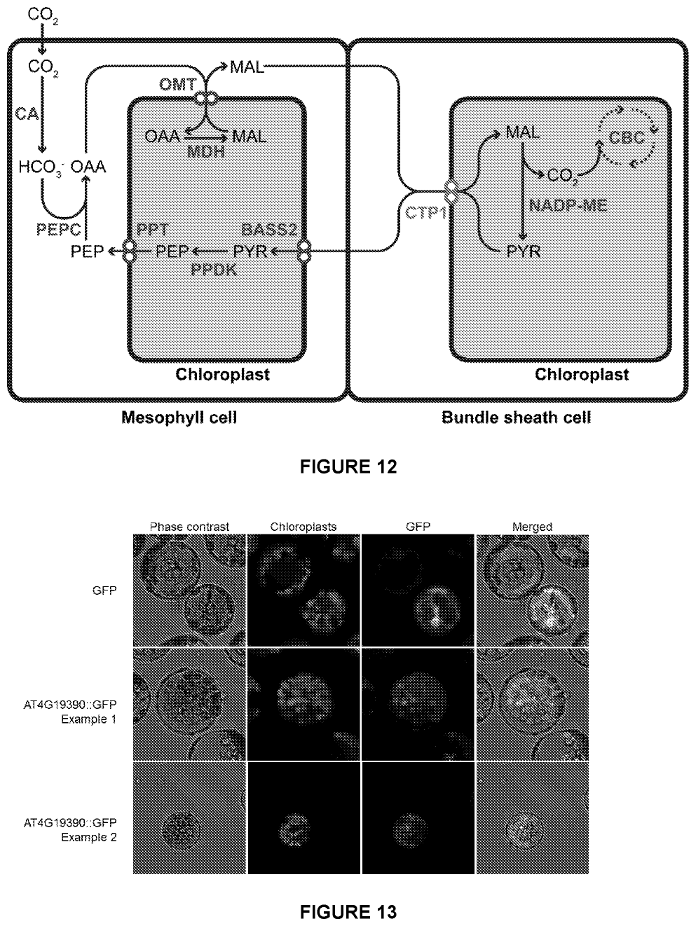

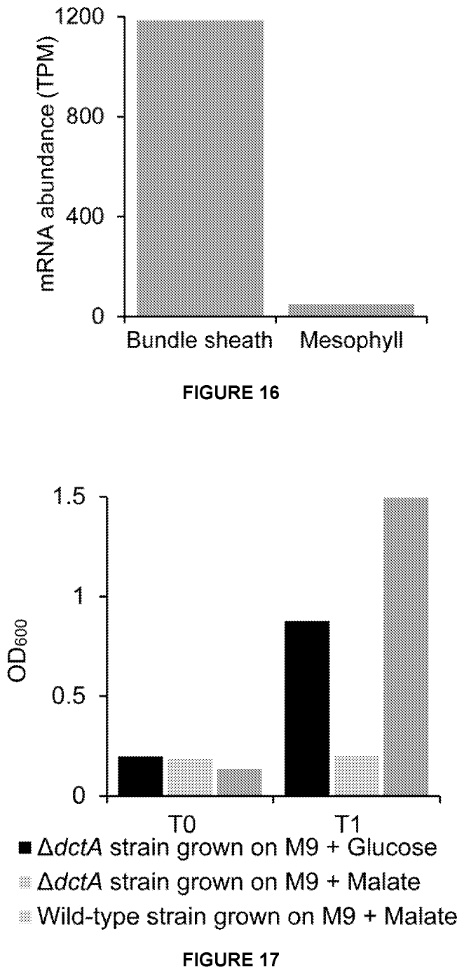

As the protein encoded by the Setaria italica representative member of this gene family can uptake malate and export pyruvate, and as it localises to the chloroplast envelope, and as it is extremely highly expressed in bundle sheath cells of the C₄ plant Setaria viridis (FIG. 16), it was proposed that the transporter provides both the malate uptake function (FIG. 2) and pyruvate export function (FIG. 2) of the bundle sheath chloroplast in a single protein (FIG. 12). To demonstrate the role for the transporter in C₄ photosynthesis an RNAi construct was generated to target the knockdown the ortholog of the transporter in Setaria viridis (Gene I.D. Sevir.4G287300, SEQ ID NO: 6). Setaria viridis is a C₄ plant that is a close relative of Setaria italica. The nucleotide sequence used for the RNAi fragment is shown in SEQ ID NO: 17. The pANIC 12A vector containing two copies of the RNAi fragment in opposite orientations separated by a GUS\linker is shown in SEQ ID NO: 15.

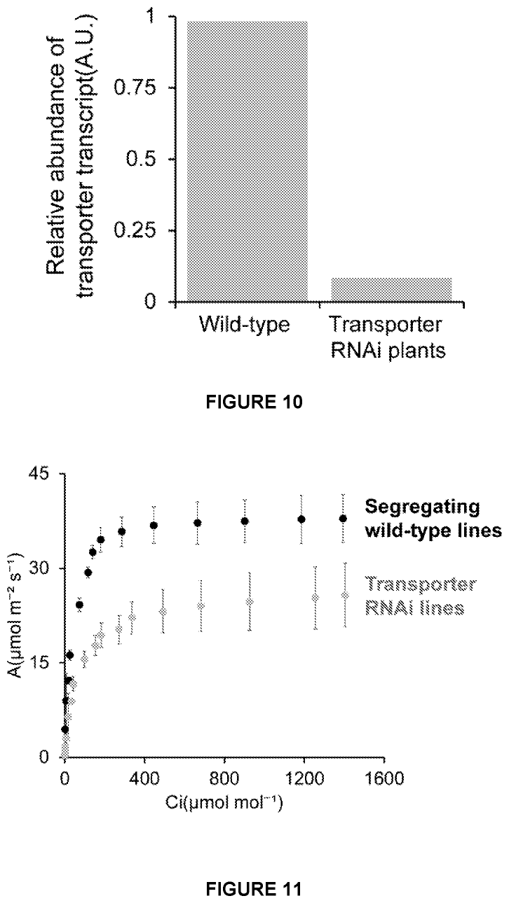

The construct was transformed into callus generated from the Setaria viridis ME034V ecotype. Transgenic plants were screened by PCR for presence of insert in TO generation. Plants that were positive for the selectable marker gene and for the RNAi fragment were taken forward for screening my quantitative PCR. T0 plants with low levels of expression of the Setaria viridis gene Sevir.4G287300 were selected. Plants had ˜10% levels of expression of the gene compared to wild-type plants (FIG. 10).

Knock-down plants were subject to photosynthesis phenotyping using a LI-COR LI-6800 to measure photosynthetic rate. Photosynthetic response to CO₂ concentration curves (also known as CO₂ response curves or A/Cᵢ curves) were conducted. This revealed that knock-down of the transporter severely disrupted C₄ photosynthesis (FIG. 11). Thus, reduction of the malate and pyruvate transport functions caused by the reduction in expression of the transporter gene cause a dramatic reduction in photosynthesis in C₄ plants. Thus, this transporter provides the malate import and pyruvate export functions of bundle sheath chloroplasts (FIG. 12).

### Example Seven

### Pyruvate Efflux Activity Can be Stimulated by the Presence of Exogenous Malate

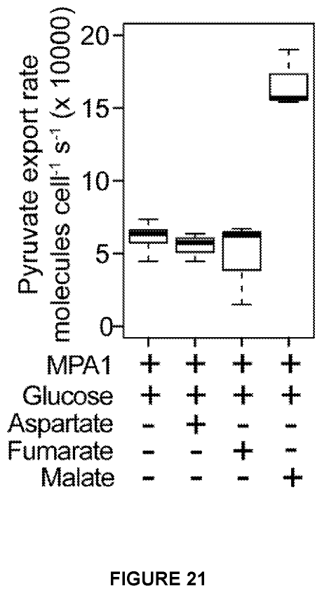

The import of malate and efflux of pyruvate from cells expressing members of the UPF0114 gene family is compatible with the hypothesis that the proteins of this family can function as antiporters. A key prediction of this hypothesis that E. coli cells expressing any member of this gene family, when fed on glucose, will show a rapid and substantial increase in pyruvate efflux if malate (and not other dicarboxylates) is added to the cell culture medium. To test this prediction, E. coli AdctA cells were grown on glucose, then expression of the Setaria italica Seita.4G275500 gene (SEQ ID NO: 8) was induced, different four-carbon dicarboxylates were added to the cell culture medium, and rapid changes to pyruvate efflux rate were assessed. Stimulated pyruvate efflux was only detected in cells that were supplemented with exogenous malate (FIG. 21) and not with other four-carbon dicarboxylates such as aspartate or fumarate (FIG. 21). Thus, members of the UPF0114 gene family can function as antiporters.

### Example Eight

### Members of the UPF0114 Gene Family are Highly Expressed in Plants that Conduct CAM Photosynthesis.

As well as being key metabolites of the C₄ photosynthetic pathway, pyruvate and malate are also key metabolites of CAM photosynthesis. In the CAM photosynthetic pathway malate is biosynthesised and accumulated during the night and then decarboxylated during the day. This process stores CO₂ at night and releases it during the day to enhance CO₂ concentration around RuBisCO. This process enhances the water use efficiency of the plant as it allows the plants to shut their stomata during the day and thus reduce water loss through transpiration.

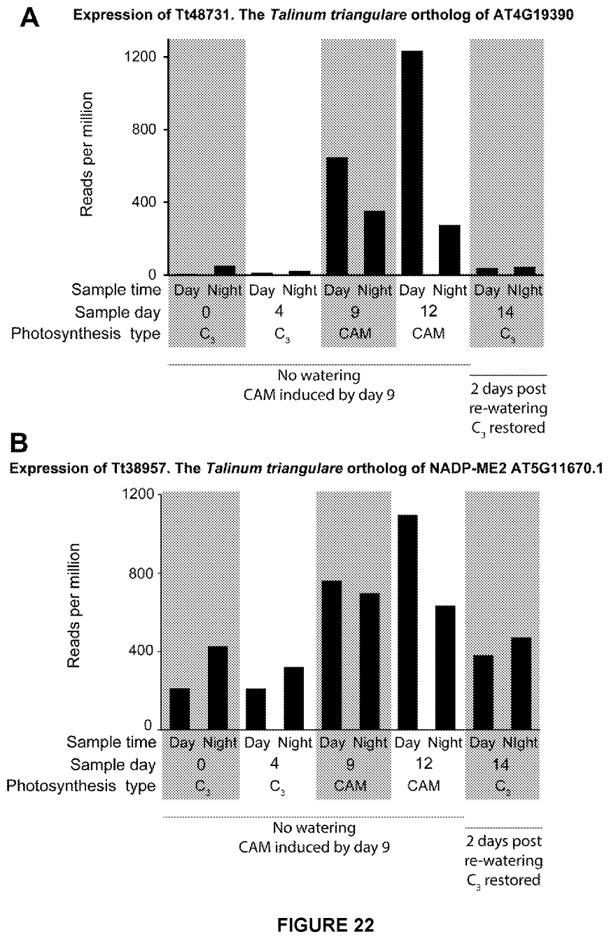

Several species of plant perform inducible CAM photosynthesis whereby they can switch between C₃ and CAM photosynthesis depending on conditions. Under well-watered growth conditions these plants perform normal C₃ photosynthesis. However, under drought conditions or, when water is scarce, these plants switch to using CAM photosynthesis to improve their water use efficiency. Accordingly, there are two hallmarks that characterise genes that are involved in the CAM photosynthetic pathway. 1) The transcripts corresponding to the genes show a substantial increase in abundance when plants switch from C₃ to CAM photosynthesis and the CAM pathway becomes active. 2) When conducting CAM photosynthesis, the transcripts corresponding to the genes differentially accumulate in between the day and the night. Transcriptome analysis of two different inducible CAM plants species demonstrate that the members of the UPF0114 gene family display both of these hallmarks of functioning in CAM photosynthesis. Specifically, analysis of the transcriptome of Talinum triangulare (Brilhaus et al. 2016. Plant Physiology 170(1) 102-122) revealed that the transcripts corresponding to the ortholog of AT4G19390 in Talinum triangulare (Tt48731, SEQ ID NOs 15 and 16) substantially increase in abundance when the plant switches from C₃ to CAM photosynthesis (FIG. 22A). In support of this specific role in CAM photosynthesis, the transcripts corresponding to the Tt48731 gene in Talinum triangulare substantially decrease in abundance when water is provided and the plant switches back to conducting C₃ photosynthesis (FIG. 22A). Thus, the gene is only highly expressed when the plant conducts CAM photosynthesis and not C₃ photosynthesis. Furthermore, when the gene is expressed it shows the second hallmark of functionality in CAM photosynthesis, namely it is differentially expressed between the day and the night (FIG. 22A). Here, it shows substantially higher expression during the day when malate is decarboxylated to pyruvate. This expression pattern is similar to the expression pattern of NADP-ME, the chloroplast localised NADP-malic enzyme responsible for decarboxylating malate in the chloroplast (FIG. 22B). The expression of the chloroplast targeted NADP-ME is induced when the plants switch to CAM photosynthesis, and NADP-ME is more highly expressed during the day than during the night (FIG. 22B). Thus, the Talinum triangulare transporter encoded by the Tt48731 gene also functions to transport malate and pyruvate into and out of the chloroplast during CAM photosynthesis. The ortholog of AT4G19390 in Mesembryanthemum crystallinum, a different inducible CAM species, also shows 29-fold upregulation to become one of the top 30 might highly upregulated genes when the plants switch from C₃ to CAM photosynthesis (Cushman et al. Journal of Experimental Botany, Volume 59, Issue 7, May 2008, Pages 1875-1894). Thus, this transporter functions in multiple different CAM species.

### INCORPORATION BY CROSS REFERENCE

The present application claims priority from Australian provisional patent application number 2019902940, the entire contents of which are incorporated herein by cross-reference.

1. A recombinant cell engineered to overexpress a UPF0114 family protein as compared to a corresponding wild-type form of the cell, wherein the UPF0114 family protein is encoded by a recombinant nucleic acid sequence stably or transiently introduced into the recombinant cell, and is capable of transporting carboxylates and/or carboxylic acids across a membrane of the recombinant cell.

2. The recombinant cell of claim 1, wherein:
the carboxylates comprise any one of:
(i) monocarboxylates;
(ii) dicarboxylates; or
(iii) tricarboxylates; or
(iv) monocarboxylates and dicarboxylates; or
(v) monocarboxylates and tricarboxylates; or
(vi) dicarboxylates and tricarboxylates; or
(vii) monocarboxylates, dicarboxylates and tricarboxylates;
the carboxylic acids comprise any one of:
(i) monocarboxylic acids;
(ii) dicarboxylic acids; or
(iii) tricarboxylic acids; or
(iv) monocarboxylic acids and dicarboxylic acids; or
(v) monocarboxylic acids and tricarboxylic acids; or
(vi) dicarboxylic acids and tricarboxylic acids; or
(vii) monocarboxylic acids, dicarboxylic acids and tricarboxylic acids.

3. The recombinant cell of claim 1, wherein:
(i) the corresponding wild-type form of the cell does not express the UPF0114 family protein; or
(ii) the UPF0114 family protein is exogenous to the recombinant cell; or
(iii) the carboxylates comprise any one or more of: malate, pyruvate, succinate, fumarate, α-ketoglutarate, citrate, glycerate-3-phosphate, phosphoenolpyruvate; or
(iv) the carboxylic acids comprise any one or more of: malic acid, pyruvic acid, succinic acid, fumaric acid, α-ketoglutaric acid, citric acid, 3-phosphoglyceric acid, phosphoenolpyruvic acid.

4. (canceled)

5. (canceled)

6. The recombinant cell of claim 1, wherein the UPF0114 family protein is capable of bidirectional transport of the carboxylates and/or carboxylic acids across the membrane.

7. (canceled)

8. The recombinant cell of claim 1, wherein the membrane is selected from a cytoplasmic membrane, a cell-internal membrane, a chloroplast membrane, an inner chloroplast envelope membrane, an outer chloroplast envelope membrane, a chloroplast internal membrane, a thylakoid membrane, a peroxisomal membrane, a mitochondrial membrane, an inner mitochondrial membrane, or an outer mitochondrial membrane.

9. The recombinant cell of claim 1, wherein the UPF0114 family protein is capable of transporting carboxylates and/or carboxylic acids across a membrane of the recombinant cell against a concentration gradient existing on one side of the membrane.

10. (canceled)

11. The recombinant cell of claim 1, wherein the recombinant cell is:
(i) a prokaryotic, eukaryotic, archaeal, plant, algal, bacterial, yeast, fungal, animal, mammalian, or synthetic cell; or
(ii) a recombinant Corynebacterium species, a recombinant Xanthomonas species, a recombinant Escherichia species, a recombinant Bacillus species, a recombinant Clostridium species, a recombinant Lactobacillus species, a recombinant Lactococcus species, a recombinant Streptococcus species, a recombinant Actinomycetes species, a recombinant Streptomyces species, or a recombinant Actinobacillus species; or
(iii) a recombinant Escherichia coli cell; or
(iv) a plant cell or an algal cell; or
(v) a plant cell that is : a vascular sheath cell, a bundle sheath cell, a mestome sheath cell, or a mesophyll cell; of a C₃ photosynthetic plant, a CAM photosynthetic plant, or a C₄ photosynthetic plant.

12. (canceled)

13. (canceled)

14. The recombinant cell of claim 11, wherein:
the carboxylates comprise any one or more of: succinate, pyruvate, fumarate, malate, citrate, phosphoenolpyruvate, α-ketoglutarate, 3-phosphoglycerate; or
the carboxylic acids comprise any one or more of: succinic acid, pyruvic acid, fumaric acid, malic acid, citric acid, phosphoenolpyruvic acid, α-ketoglutaric acid, 3-phosphoglyceric acid.

15. (canceled)

16. The recombinant cell of claim 11, wherein the recombinant cell is a plant cell and the plant cell is: a vascular sheath cell, a bundle sheath cell, a mestome sheath cell, or a mesophyll cell; of a C₃ photosynthetic plant, a CAM photosynthetic plant, or a C₄ photosynthetic plant.

17. The recombinant cell of claim 11, wherein the recombinant cell is a plant cell, and:
the carboxylates comprise malate and/or pyruvate; or
the carboxylic acids comprise malic acid and/or pyruvic acid.

18. The recombinant cell of claim 17, wherein:
(i) the UPF0114 family protein is capable of uptaking malate and/or malic acid into the recombinant cell and exporting pyruvate and/or pyruvic acid from the recombinant cell: or
(ii) the UPF0114 family protein is capable of uptaking malate and/or malic acid into the recombinant cell and exporting pyruvate and/or pyruvic acid from the recombinant cell against a concentration gradient.

19. (canceled)

20. The recombinant cell of claim 11, wherein the recombinant cell is a plant cell and the recombinant nucleic acid sequence comprises a sequence encoding a targeting peptide targeting the UPF0114 family protein to a chloroplast membrane, a cytoplasmic membrane, a peroxisomal membrane, or a mitochondrial membrane.

21. The recombinant cell of claim 1, wherein the UPF0114 family protein comprises:
(i) a PFAM protein domain UPF0114 (PF03350) amino acid sequence as defined in any one of SEQ ID NOs: 28-37; or
(ii) a PFAM protein domain UPF0114 (PF03350) amino acid sequence having at least: 70%, 75%, 80%, 85%, 87%, 89%, 90%, 91%, 92%, 93%, 94%, 95%, 96%, 97%, 98%, 99%; sequence identity to any one of SEQ ID NOs: 28-37; or
(iii) a homolog, analog, ortholog or paralog of the PFAM protein domain UPF0114 (PF03350) amino acid sequence of (i) or (ii).

22. (canceled)

23. The recombinant cell of claim 11, wherein the recombinant cell is a plant cell and the plant cell is a genus Oryza plant (e.g. a rice plant), a Oryza sativa or Oryza glaberrima plant, or from a: Soy (Glycine max), Cotton (Gossypium hirsutum), Oilseed rape/Cannola (B. napus subsp. Napus), Potato (Solanum tuberosum), tomato (Solanum lycopersicum), Cassava (Manihot esculenta), Wheat (Triticum aestivum), Barley (Hordeum vulgare), pigeon pea (Cajanus cajan), cowpea (Vigna unguiculata), pea (Pisum sativum), cannabis (Cannabis sativa), sugar beet (Beta vulgaris), oat (Avena sativa), rye (Secale cereal), peanut (Arachis hypogaea), Sunflower (Helianthus annuus), flax (Linum spp.), beans (Phaseolus vulgaris), lima bean (Phaseolus lunatus), mung bean (Phaseolus mung), Adzuki bean (Phaseolus angularis), Chickpea (Cicer arietinum), tobacco (Nicotiana tabacum), buckwheat (Fagopyrum esculentum), oil palm (Elaeis guineensis), or rubber (Hevea brasiliensis); plant.

24. The recombinant cell of claim 1, wherein the UPF0114 family protein is:
(i) a C₄ photosynthetic plant UPF0114 protein, a C₃ photosynthetic plant UPF0114 protein, an algal UPF0114 protein, a bacterial UPF0114 protein, or an archaeal UPF0114 protein; or
(ii) an Arabidopsis thaliana UPF0114 protein; or
(ii) a Setaria italica UPF0114 protein; or
(iii) a Setaria viridis UPF0114 protein; or
(iv) an Escherichia coli UPF0114 protein; or
(v) a Zea mays UPF0114 protein; or
(vi) a UPF0114 protein comprising or consisting of an amino acid sequence having at least:
70%, 75%, 80%, 85%, 87%, 89%, 90%, 91%, 92%, 93%, 94%, 95%, 96%, 97%, 98%, 99%; sequence identity to the UPF0114 protein of (i), (ii), (iii), (iv) or (v); or
(vii) a homolog, analog, ortholog or paralog of the UPF0114 protein of (i), (ii), (iii), (iv) or (v).

25. (canceled)

26. The recombinant cell of claim 1, wherein the UPF0114 family protein:
(i) comprises or consists of an amino acid sequence as defined in SEQ ID NO: 1, SEQ ID NO: 2, SEQ ID NO: 3, SEQ ID NO: 4, SEQ ID NO: 5, SEQ ID NO: 6; SEQ ID NO: 9, SEQ ID NO: 10, SEQ ID NO: 11, SEQ ID NO: 15, SEQ ID NO: 18, SEQ ID NO: 19, SEQ ID NO: 20, SEQ ID NO: 21, SEQ ID NO: 212, SEQ ID NO: 23, SEQ ID NO: 24, SEQ ID NO: 25, SEQ ID NO: 26, or SEQ ID NO: 27; or
(ii) comprises or consists of an amino acid sequence having at least: 70%, 75%, 80%, 85%, 87%, 89%, 90%, 91%, 92%, 93%, 94%, 95%, 96%, 97%, 98%, 99%; sequence identity to SEQ ID NO: 1, SEQ ID NO: 2, SEQ ID NO: 3, SEQ ID NO: 4, SEQ ID NO: 5, SEQ ID NO: 6 SEQ ID NO: 9, SEQ ID NO: 10, SEQ ID NO: 11, SEQ ID NO: 15, SEQ ID NO: 18, SEQ ID NO: 19, SEQ ID NO: 20, SEQ ID NO: 21, SEQ ID NO: 212, SEQ ID NO: 23, SEQ ID NO: 24, SEQ ID NO: 25, SEQ ID NO: 26, or SEQ ID NO: 27; or
(iii) is a homolog, analog, ortholog or paralog of the UPF0114 family protein comprising or consisting of an amino acid sequence of (i) or (ii); or
(iv) is encoded by a nucleotide sequence comprising or consisting of SEQ ID NO: 7, SEQ ID NO: 8, SEQ ID NO: 12, SEQ ID NO: 13, SEQ ID NO: 14, or SEQ ID NO: 16; or
(v) is encoded by a nucleotide sequence comprising or consisting a nucleotide sequence having at least: 70%, 75%, 80%, 85%, 87%, 89%, 90%, 91%, 92%, 93%, 94%, 95%, 96%, 97%, 98%, 99%; sequence identity to SEQ ID NO: 7 SEQ ID NO: 8, SEQ ID NO: 12, SEQ ID NO: 13, SEQ ID NO: 14, or SEQ ID NO: 16; or
(vi) is a homolog, analog, ortholog or paralog of the UPF0114 family protein encoded by the nucleotide sequence of (iv) or (v).

27. The recombinant cell of claim 1, wherein the recombinant nucleic acid sequence:
(i) is operably linked to a regulatory sequence; and/or
(ii) is a component of an expression vector; and/or
(iii) is codon optimised for expression in the recombinant cell type; and/or
(iv) has intronic sequences removed; and/or
(v) comprises a signal peptide sequence for directing the UPF0114 family protein to an internal membrane or cytoplasmic membrane of the recombinant cell.

28. The recombinant cell of claim 1, wherein:
(i) the carboxylates and/or carboxylic acids are phosphorylated; or
(ii) the recombinant cell is further engineered to produce or overexpress an enzyme and/or regulatory protein of a biochemical pathway, for production of the carboxylates and/or carboxylic acids.

29. (canceled)

30. (canceled)

31. A transgenic plant or a seed thereof comprising the recombinant plant cell of claim 11.

32. (canceled)

33. (canceled)

34. A process for production of carboxylic acids and/or carboxylates comprising:
(i) producing the carboxylates in the recombinant cell according to claim 1, and
(ii) exporting the carboxylates from the recombinant cell using a UPF0114 family protein embedded within the membrane of the recombinant cell.

35. (canceled)

36. (canceled)

37. (canceled)

38. (canceled)

39. (canceled)

40. (canceled)


SEQUENCE LISTING (37 sequences)

SEQ ID NO: 1 - protein, 164 aa, Escherichia coli
MERFLENAMYASRWLLAPVYFGLSLALVALALKFFQEIIHVLPNIFSMAESDLILVLLSLVDMTLVGGLLVMVMFSGYENFVSQLDISENKEKLNWLGKMDATSLKNKVAASIVAISSIHLLRVFMDAKNVPDNKLMWYVIIHLTFVLSAFVMGYLDRLTRHNH

SEQ ID NO: 2 - protein, 273 aa, Arabidopsis thaliana
MTTPCRTINANAIAAPSPSGLIFNGFRDFVPIEKRLVISSFRGLKLPSRTTKTITSSDWSWSYRSPGRLASASTSTSASTSTSAAVTSNSTNRFEALEEGIEKVIYSCRFMTFLGTLGSLLGSVLCFIKGCMYVVDSFLQYSVNRGKVIFLLVEAIDIYLLGTVMLVFGLGLYELFISNLDTSESRTHDIVSNRSSLFGMFTLKERPQWLEVKSVSELKTKLGHVIVMLLLIGLFDKSKRVVITSVTDLLCISVSIFFSSACLFLLSRLNGSH

SEQ ID NO: 3 - protein, 247 aa, Setaria italica
MKLRPLTCVAAGCAGWAWRPRSRVRSEAVSPKRSHAAAAAAGAVHSEEHRRGGMREVLFRPVGLPTETKFGAGLEDRIEKVICACRFMTFLGIGGLLAGCVPCFLKGCVYVMDAFVEYYLHGGGMLILMLLEAIDMFLIGTVMFVFGTGLYELFISEMDMSYGSNLFGLFSLPERPKWLVIQSVNDLKTKLGHVIVMSLLVGIFEKSWRVTITSCTDLLCFAASIFLSSGCLYLLSRLSNTKGGSHT

SEQ ID NO: 4 - protein, 185 aa, Artificial Sequence
  Codon optimised version of Arabidopsis thaliana AT4G19390 protein with no chloroplast target peptide
MSTNRFEALEEGIEKVIYSCRFMTFLGTLGSLLGSVLCFIKGCMYVVDSFLQYSVNRGKVIFLLVEAIDIYLLGTVMLVFGLGLYELFISNLDTSESRTHDIVSNRSSLFGMFTLKERPQWLEVKSVSELKTKLGHVIVMLLLIGLFDKSKRVVITSVTDLLCISVSIFFSSACLFLLSRLNGSH

SEQ ID NO: 5 - protein, 247 aa, Artificial Sequence; identical to SEQ ID NO: 3
  Codon optimised version of Setaria italica Si007164m (Seita.4G275500) protein with no chloroplast target peptide

SEQ ID NO: 6 - protein, 247 aa, Setaria viridis; identical to SEQ ID NO: 3

SEQ ID NO: 7 - DNA, 558 bp, Artificial Sequence
  Codon optimised version of Arabidopsis thaliana AT4G19390 gene with no chloroplast target peptide
atgagtaccaaccgttttgaagccttagaggaagggattgaaaaagttatttattcgtgtcgttttatgacgttcttaggtacactggggtccttgttaggtagcgtgctgtgtttcatcaagggctgtatgtatgttgtagattcttttcttcaatattctgtcaatcgcgggaaggttattttcctgttggtcgaggccattgatatttatttgttgggaaccgttatgttagtgtttggactgggcctgtacgagctgttcatctcgaatctggatacttctgagagccgcacccacgacatcgtttctaatcgctcatccttgtttggtatgttcaccttgaaggagcgcccccaatggcttgaagtaaaatcggtgagcgagctgaaaacgaaactgggtcacgtaattgttatgttgttactgatcgggttatttgataagtctaaacgtgttgttatcaccagtgttacggacctgttatgcattagtgtaagcatcttcttcagctcagcatgtctgttcttgttaagccgtcttaacggcagccactga

SEQ ID NO: 8 - DNA, 744 bp, Artificial Sequence
  Codon optimised version of Setaria italica Si007164m (Seita.4G275500) gene with no chloroplast target peptide
atgaagctcaggcctctcacttgcgtggcggcggggtgcgccgggtgggcgtggaggccgaggtcgcgcgtgcggtcagaggcggtgtcacccaagcgttcccacgcggcagcggcggcggcgggcgcggttcattcggaggagcaccgccgcggcggcatgcgcgaggtgctcttccgcccggtggggctgcccaccgagacgaagttcggggcggggctggaggatcggatcgagaaggtcatctgcgcctgccgcttcatgaccttcctcggcatcggcggcttgctcgccggctgcgtcccctgcttcctcaagggatgcgtttatgtgatggacgccttcgtcgagtactacctgcacggcggtggaatgctcatcctaatgttgcttgaagccattgacatgtttctcattggaacggtcatgtttgtattcgggacgggcttgtatgagctgttcatcagtgaaatggacatgtcttatggctccaacttgtttggcttgttcagtcttccggaacgacccaagtggctggtaatccagtcggtgaatgatcttaagacaaagctgggccatgtcattgtcatgagtctactggttggcatctttgagaagagctggagagtgaccattacatcctgtactgacctcctttgcttcgctgcatcaatcttcctctcctcaggttgcctctacctactttccaggctcagtaacaccaaaggagggagccatacctga

SEQ ID NO: 9 - protein, 308 aa, Zea mays
MAGRREPRSPSIMLRPGQRRRNYLRRHPPLTTGPGADEMNGNGCPSPPPTWTRCLPRKAPRPLGCGCGCVPAAVGCVGWAWRPTPRPRGGGRAAGVSPKCSHSAAAAGAVQSEDRRREVLYRPVELPGTGYGSELEARIEKVIYACRFMTFFGICGLLLGSVPCFLKGCVFVMDAFVEYYRHGAGKVILLLVEAIEMFLIATVTFVLGTGLYELFISNMDSFYGSNLFGLFSLPERPKWVEIKSVNDLKTKLGHVIVMVLLVGIFEKSKRVTITSCADLLCFAGSIFLSSVCLYLLSKLHTTKGGSQA

SEQ ID NO: 10 - protein, 266 aa, Zea mays
MALLLLRGCAAPPAVHAAPAGSRLLPPALPRRRLVAVASSASPAPSGEVASSSQDGRGYGTVGGPNGHAIAPATVTKSTAVETTVERVIFDFRFLALLAVAGSLAGSVLCFLNGCVFIKEAYQVYWSSCVKGVHTGQMVLKVVEAIDVYLAGTVMLIFGMGLYGLFISNAPASVAPESDRALSGSSLFGMFALKERPKWMNITSLDELKTKVGHVIVMILLVKMFEKSKMVTIATGLDLLSYSICIFLSSASLYILHNLHKGDHEE

SEQ ID NO: 11 - protein, 262 aa, Zea mays
MALLVLRAPAAVHAASRLLPPQPRRRRRLVAVASAASSAPSGEVSSQHGGGGGGGYGIVGGPNGNAVVPATKSTVVETTVERVIFDFRFLALLAVAGSLAGSLLCFLNGCVFIKEAYQVYWSSCVKGVHTGQMVLKVVEAIDVYLAGTVMLIFGMGLYGLFVSNASAGVGSESDRALSGSSLFGMFALKERPKWMKITSLDELKTIVGHVIVMILLVKMFERSKMVTIATGLDLLSYSICIFLSSASLYILHNLHKGDDHEE

SEQ ID NO: 12 - DNA, 525 bp, Artificial Sequence
  Codon optimised version of Zea mays GRMZM2G179292 gene with no chloroplast target peptide
atggaagcccgcattgagaaagtcatatacgcgtgccggtttatgaccttttttggtatttgtggcctgctgctgggatcggttccatgcttcctgaaaggctgtgtgttcgtaatggatgcatttgtggagtactatcgtcatggtgcaggtaaagtgattctgctgctggtcgaggccatcgaaatgttcttgatcgctactgtcacatttgtgttgggtacgggcctgtacgaacttttcatcagcaacatggattccttttatgggagtaacctttttgggcttttctccctgccggaacgccctaaatgggtagaaatcaaatccgttaatgacttgaaaactaaacttggtcacgtgatcgttatggttctgttagtgggaatctttgaaaagtcgaagcgtgtcactatcacgtcctgcgcggatttactttgctttgcgggctctatcttcttgagctcagtatgtctgtatttgcttagcaagttacatacaactaaaggaggcagtcaggcttga

SEQ ID NO: 13 - protein, 561 aa, Artificial Sequence
  Codon optimised version of Zea mays GRMZM2G133400 protein with no chloroplast target peptide
ATGGAAACGACCGTAGAACGCGTCATTTTCGATTTTCGGTTCCTGGCCCTGCTGGCGGTTGCTGGCAGCCTGGCGGGTTCTGTCCTGTGCTTTCTGAATGGTTGTGTGTTCATAAAAGAAGCCTATCAGGTTTACTGGAGCTCATGCGTGAAAGGCGTCCATACGGGTCAAATGGTGCTGAAGGTAGTAGAAGCAATCGATGTTTACTTAGCGGGGACTGTGATGCTTATTTTTGGGATGGGCTTGTATGGCCTGTTCATCTCGAACGCGCCAGCCTCGGTCGCGCCAGAATCCGACCGCGCCCTGAGCGGGAGTTCCCTGTTTGGGATGTTCGCATTAAAGGAGCGTCCAAAGTGGATGAACATCACATCTCTTGACGAGCTTAAAACCAAGGTGGGCCACGTTATTGTTATGATCTTATTAGTGAAAATGTTTGAGAAATCGAAGATGGTGACTATCGCTACCGGACTGGATCTGCTTAGCTATTCAATCTGTATCTTTTTGAGTTCCGCATCGCTTTACATCCTTCACAATTTACATAAAGGTGATCACGAAGAGTAA

SEQ ID NO: 14 - DNA, 582 bp, Artificial Sequence
  Codon optimised version of Zea mays GRMZM2G327686 gene with no chloroplast target peptide
atgacgaaaagtacagtcgtcgaaacgacggttgagcgtgttatttttgacttccgctttttagccctgttagctgtcgctggttcccttgcagggtccctgctttgttttttgaatgggtgtgtctttatcaaagaggcgtaccaagtgtattggtcgtcatgcgtaaaaggggtacatactggccagatggtcttgaaggtagtcgaggcaattgatgtttatcttgccggaaccgtaatgcttatcttcggaatgggtttgtacgggttgtttgtaagtaacgctagtgcaggggtcggtagcgaatcggatcgcgcgcttagcggaagttctcttttcgggatgtttgcccttaaagaacgcccgaagtggatgaaaatcacctcactggacgagttaaagacgattgttggtcatgtgatcgttatgattcttttggtgaagatgtttgaacgtagtaaaatggtaactattgcgaccggattggacttacttagctattcgatttgcatctttttaagcagtgcaagcctgtatatcctgcacaacctgcataagggcgacgatcacgaggaataa

SEQ ID NO: 15 - DNA, 792 bp, Talinum triangulare
atgaagacactcaaagctcatcagttcttgctatcttctcccaaacccacatcgtttatcctcggaaaaccctcgaggaatatgaggttgaggaccccattgacgcgtcgattcagggcgtgtcggacggatcagatttcggctccgagtaagattgcggcgccaaatggttcttcctcttcgtccctaatggctcccggcggggggtctaccgggttccggcgtcgtgtttgggtgtctgaatctatggaggaagctcttgaaaaggctatttatcggtctcggttcatgacgcttcttggagttttaggctctttggtgggatctgttctctgcttcgtcaagggttgtaatattgtggcagcttctttcactgagcacattgtaaggagcgggaaggtgatgactgtgctggttgaggctttagatgtttatctgcttggaacggtgatgctggtatttggaatggggctttatgagctatttgtgtgcaatattgacattgaagagtcactgaaaggtcaaaaatttccttatcggtcaaatttgtttggcttgttcactttaatggaacggccgaaatggttggagataaagtcagtcaatgagctgaagactaaggttggacatgtaatagtgatgctgttgctgataggattctttgacaatagtaagaaagcagctattcactctcctacagatttactctgcttctcagcctccattctcctttgctcaggttgcctttacttgctggctaagctcaatggccctaagcatcaatggctctaa

SEQ ID NO: 16 - protein, 263 aa, Talinum triangulare
MKTLKAHQFLLSSPKPTSFILGKPSRNMRLRTPLTRRFRACRTDQISAPSKIAAPNGSSSSSLMAPGGGSTGFRRRVWVSESMEEALEKAIYRSRFMTLLGVLGSLVGSVLCFVKGCNIVAASFTEHIVRSGKVMTVLVEALDVYLLGTVMLVFGMGLYELFVCNIDIEESLKGQKFPYRSNLFGLFTLMERPKWLEIKSVNELKTKVGHVIVMLLLIGFFDNSKKAAIHSPTDLLCFSASILLCSGCLYLLAKLNGPKHQWL

SEQ ID NO: 17 - DNA, 461 bp, Artificial Sequence
  RNAi targetting Setaria viridis Sevir.4G287300 gene.
atgaagctcaggcctctcacttgcgtggcggcggggtgcgccgggtgggcgtggaggccgaggtcgcgcgtgcggtcagaggcggtgtcacccaagcgttcccacgcggcagcggcggcggcgggcgcggttcattcggaggagcaccgccgcggcggcatgcgcgaggtgctcttccgcccggtggggctgcccaccgagacgaagttcggggcggggctggaggatcggatcgagaaggtcatctgcgcctgccgcttcatgaccttcctcggcatcggcggcttgctcgccggctgcgtcccctgcttcctcaagggatgcgtttatgtgatggacgccttcgtcgagtactacctgcacggcggtggaatgctcatcctaatgttgcttgaagccattgacatgtttctcattggaacggtcatgtttgtattcgggacgggcttgtatgagctgtt

SEQ ID NO: 18 - protein, 177 aa, Caulobacter phage
MIFETRWLLVPIYLAMIIAIAAYVILFTKQAIDMGLGVWHWDAEHLLLASLALVDMSMVANLIVMILAGGFSTFVAEFDQSLFPNRPRWMNGLDSTTLKIQMGKSLIGVTSVHLLQTFMRLHDILKEENGLVLVIAEIAIHMVFIVTTVSYCYISKLTHGHKVAPAALPTPATAEGH

SEQ ID NO: 19 - protein, 186 aa, Methanosarcina spelaei
MKVVRFIAGMRFFVLIPVIGLAIAACVLFIKGGIDIIHFMGELIIGMSEEGPEKSIIVEIVETVHLFLVGTVLFLTSFGLYQLFIQPLPLPEWVKVNNIEELELNLVGLTVVVLGVNFLSIIFEPQETDLAIYGIGYALPIAALAYFMKVRSHIRKGSNDEEEMRNIGEVTSVNSESNWLINKKGD

SEQ ID NO: 20 - protein, 185 aa, Methanococcus maripaludis
MGKSDKLKKKYGIKNISEQGFFEHFFELILWNSRFIVVLAVIFGTLGSIMLFLAGSAEIFHTILSYISDPMSSEQHNQILIGVIGAVDLYLIGVVLLIFSFGIYELFISKIDIARVDGDVSNILEIYTLDELKSKIIKVIIMVLVVSFFQRVLSMHFETSLDMIYMAISIFAISLGVYFMHRQKM

SEQ ID NO: 21 - protein, 164 aa, Escherichia coli; identical to SEQ ID NO: 1

SEQ ID NO: 22 - protein, 168 aa, Campylobacter concisus
MRKIFERILLASNSFTLFPVVFGLLGAIVLFIIASYDVGKVLLEVYKYFFAADFHVENFHSEVVGEIVGAIDLYLMALVLYIFSFGIYELFISEITQLKQSKQSKVLEVHSLDELKDKLGKVIVMVLIVNFFQRVLHANFTTPLEMAYLAASILALCLGLYFLHKGDH

SEQ ID NO: 23 - protein, 170 aa, Rhodobacteraceae bacterium
MGFIERIGEKILWNSRFIVILAVIFSIIASISLFIIGSYEIIYSLVYENPIWSEKYKHNHAQILYKIISAVDLYLIGVVLMIFGFGIYELFISKIDIARKNPSITILEIENLDELKNKIVKVIVMVLIVSFFERILKNSDAFTSSLNLLYFAISIFAISFSIYYINKNKN

SEQ ID NO: 24 - protein, 302 aa, Micromonas pusilla
MSSSGVLSLSASARVAPRATSVRRARAPVRATQLARSRADTAAWGKKFMSVERGSRAVGVRSLVEAANTEPGASYDDGDDHVDTTYDAEDLAHPDVAMMKASREVRKPFREFSLIEKVEYVFVRFTLISACIFVLLGVLASLLLSALLFSMGMKEVLFDAVQAWAGYSPVGLVSSAVGALDRFLLGMVCLVFGLGSFELFLARSNRAGQVRDRRLKKLAWLKVSSIDDLEQKVGEIIVAVMVVNLLEMSLHMTYAAPLDLVWAALAAVMSAGALALLHYAAGHGDHNHKDKGGHDSGAGLLH

SEQ ID NO: 25 - protein, 232 aa, Klebsormidium nitens
MSKDGVAAIDVMMPDGASEDYPITLEEADASDGEWTRRKRHVKRLKKVESTIERVIFDCRFFALMGVVGSLIGSFLCFVKGCFYVYKAIIAAAFDVTHGLNSYKVVLKLIEALDTYLVATVMLIFGMGLYELFVNELEAVATTDSVVGCKSNLFGLFRLRERPKWLQINGLDALKEKLGHVIVMILLVGMFEKSKKVPIRNGVDLVCVATSVLLCAGSLYLLSQLSKNGNGH

SEQ ID NO: 26 - protein, 262 aa, Arabidopsis thaliana
MALSSLISATPLSLSVPRYLVLPTRRRFHLPLATLDSSPPESSASSSIPTSIPVNGNTLPSSYGTRKDDSPFAQFFRSTESNVERIIFDFRFLALLAVGGSLAGSLLCFLNGCVYIVEAYKVYWTNCSKGIHTGQMVLRLVEAIDVYLAGTVMLIFSMGLYGLFISHSPHDVPPESDRALRSSSLFGMFAMKERPKWMKISSLDELKTKVGHVIVMILLVKMFERSKMVTIATGLDLLSYSVCIFLSSASLYILHNLHKGET

SEQ ID NO: 27 - protein, 344 aa, Oryza sativa
MAAAAAGGGGGGGGSGRLLRGATAKAFHGDGSSHHRMMPSSSSSVAAGGGGGVAGPCRIPSLKFPSLWESKRQGGGVGSRAAERKAALIALGAAGVTALERERGGGVVLLPEEARRGADLLLPLAYEVARRLVLRQLGGATRPTQQCWSKIAEATIHQGVVRCQSFTLIGVAGSLVGSVPCFLEGCGAVVRSFFVQFRALTQTIDQAEIIKLLIEAIDMFLIGTALLTFGMGMYIMFYGSRSIQNPGMQGDNSHLGSFNLKKLKEGARIQSITQAKTRIGHAILLLLQAGVLEKFKSVPLVTGIDMACFAGAVLASSAGVFLLSKLSTTAAQAQRQPRKRTAFA

SEQ ID NO: 28 - protein, 138 aa, Caulobacter phage
IFETRWLLVPIYLAMIIAIAAYVILFTKQAIDMGLGVWHWDAEHLLLASLALVDMSMVANLIVMILAGGFSTFVAEFDQSLFPNRPRWMNGLDSTTLKIQMGKSLIGVTSVHLLQTFMRLHDILKEENGLVLVIAEIA

SEQ ID NO: 29 - protein, 145 aa, Methanosarcina spelaei
VVRFIAGMRFFVLIPVIGLAIAACVLFIKGGIDIIHFMGELIIGMSEEGPEKSIIVEIVETVHLFLVGTVLFLTSFGLYQLFIQPLPLPEWVKVNNIEELELNLVGLTVVVLGVNFLSIIFEPQETDLAIYGIGYALPIAALAYF

SEQ ID NO: 30 - protein, 159 aa, Methanococcus maripaludis
FEHFFELILWNSRFIVVLAVIFGTLGSIMLFLAGSAEIFHTILSYISDPMSSEQHNQILIGVIGAVDLYLIGVVLLIFSFGIYELFISKIDIARVDGDVSNILEIYTLDELKSKIIKVIIMVLVVSFFQRVLSMHFETSLDMIYMAISIFAISLGVYFM

SEQ ID NO: 31 - protein, 150 aa, Escherichia coli
ERFLENAMYASRWLLAPVYFGLSLALVALALKFFQEIIHVLPNIFSMAESDLILVLLSLVDMTLVGGLLVMVMFSGYENFVSQLDISENKEKLNWLGKMDATSLKNKVAASIVAISSIHLLRVFMDAKNVPDNKLMWYVIIHLTFVLSAF

SEQ ID NO: 32 - protein, 165 aa, Campylobacter concisus
KIFERILLASNSFTLFPVVFGLLGAIVLFIIASYDVGKVLLEVYKYFFAADFHVENFHSEVVGEIVGAIDLYLMALVLYIFSFGIYELFISEITQLKQSKQSKVLEVHSLDELKDKLGKVIVMVLIVNFFQRVLHANFTTPLEMAYLAASILALCLGLYFLHKGD

SEQ ID NO: 33 - protein, 162 aa, Rhodobacteraceae bacterium
ERIGEKILWNSRFIVILAVIFSIIASISLFIIGSYEIIYSLVYENPIWSEKYKHNHAQILYKIISAVDLYLIGVVLMIFGFGIYELFISKIDIARKNPSITILEIENLDELKNKIVKVIVMVLIVSFFERILKNSDAFTSSLNLLYFAISIFAISFSIYYIN

SEQ ID NO: 34 - protein, 152 aa, Micromonas pusilla
TLISACIFVLLGVLASLLLSALLFSMGMKEVLFDAVQAWAGYSPVGLVSSAVGALDRFLLGMVCLVFGLGSFELFLARSNRAGQVRDRRLKKLAWLKVSSIDDLEQKVGEIIVAVMVVNLLEMSLHMTYAAPLDLVWAALAAVMSAGALALL

SEQ ID NO: 35 - protein, 174 aa, Klebsormidium nitens
ESTIERVIFDCRFFALMGVVGSLIGSFLCFVKGCFYVYKAIIAAAFDVTHGLNSYKVVLKLIEALDTYLVATVMLIFGMGLYELFVNELEAVATTDSVVGCKSNLFGLFRLRERPKWLQINGLDALKEKLGHVIVMILLVGMFEKSKKVPIRNGVDLVCVATSVLLCAGSLYLL

SEQ ID NO: 36 - protein, 174 aa, Arabidopsis thaliana
SNVERIIFDFRFLALLAVGGSLAGSLLCFLNGCVYIVEAYKVYWTNCSKGIHTGQMVLRLVEAIDVYLAGTVMLIFSMGLYGLFISHSPHDVPPESDRALRSSSLFGMFAMKERPKWMKISSLDELKTKVGHVIVMILLVKMFERSKMVTIATGLDLLSYSVCIFLSSASLYIL

SEQ ID NO: 37 - protein, 171 aa, Oryza sativa
ATIHQGVVRCQSFTLIGVAGSLVGSVPCFLEGCGAVVRSFFVQFRALTQTIDQAEIIKLLIEAIDMFLIGTALLTFGMGMYIMFYGSRSIQNPGMQGDNSHLGSFNLKKLKEGARIQSITQAKTRIGHAILLLLQAGVLEKFKSVPLVTGIDMACFAGAVLASSAGVFLLS